(19) United States
(12) Patent Application Publication
(10) Pub. No.: US 2026/0009020 A1
(43) Pub. Date: Jan. 8, 2026

(54) BEAD-HASHING

(71) Applicant: Oxford University Innovation Limited, Oxford (GB)

(72) Inventors: Martin Philpott, Oxford (GB); Adam Cribbs, Oxford (GB); Udo Oppermann, Oxford (GB)

(21) Appl. No.: 18/998,512

(22) PCT Filed: Aug. 1, 2023
(86) PCT No.: PCT/GB2023/052028

§ 371 Date: Jan. 26, 2025

(30) Foreign Application Priority Data
Aug. 1, 2022 (GB) 2211182.7

(51) Int. Cl. C12N 15/10

(52) U.S. Cl. CPC C12N 15/1065; C12N 15/1068

(57) ABSTRACT

The invention relates to means and methods for producing libraries of analytes, such as polynucleotides, from a plurality of samples, such as single cells. The invention uses micro-particles that include both barcoded analyte capture polynucleotides and barcoded polynucleotides having a hairpin sequence. The micro-particles are divided between compartments together with sample. During library production, the hairpin sequences dimerise to produce polynucleotides comprising two barcode sequences. The dimers provide information about how many micro-particles were co-compartmentalised and with which sample analytes. This bead hashing method allows for increased loading of micro-particles into compartments with sample leading to increased throughput, greater efficiency and reduced loss of sample from analysis.

### FIELD OF THE INVENTION

The disclosure relates to means and methods for bead-hashing when generating a library of analytes, such as RNA molecules, from a plurality of samples, such as single cells.

### BACKGROUND TO THE INVENTION

Single-cell sequencing is a rapidly growing field, currently lead by droplet-based methods. In these methods, individual cells are encapsulated along with oligonucleotide-barcoded RNA-capture microbeads into aqueous droplets within an oil emulsion using a micro-fluidics device. This allows thousands of cells per sample to be interrogated. However, the number of cells that can be analysed remains limited by the need to avoid forming droplets with more than one cell or bead, which confound the results. The same applies in principle to other investigations involving large numbers of samples, such as single vesicles or single nuclei, and to investigations of different types of analytes, such as RNA, DNA or proteins.

A method commonly referred to as cell hashing allows for increased detection of cells in droplet-based single-cell sequencing. In this method, cells to be sequenced are split into multiple aliquots. Each aliquot is then tagged with an antibody conjugated to a oligonucleotide barcode. Aliquots are repooled and cells super-loaded during encapsulation, resulting in more droplets with cells (but also greatly increasing droplets with >1 cell). During bioinformatic analysis, the barcodes introduced by antibody tagging allow sequencing reads resulting from droplets with more than one cell to be detected and removed. However, cell hashing involves time consuming cell preparation prior to encapsulation (which could also alter the state of the cells and create experimental artefacts) and requires additional expensive reagents. Furthermore, since the antibodies used typically tag cell surface markers, methods such as DroNc-seq (nuclei sequencing, often necessary when working with tissue samples) necessitate custom reagents.

Hence, there remains a need for new methods that increase the number of samples, such as single cells, that can be interrogated for analytes, without disproportionately increasing costs and complexity or decreasing accuracy.

### SUMMARY OF THE INVENTION

The inventors have developed a method for directly detecting multiple beads contacted with sample during analyte library preparation. The method relies on the use of modified analyte capture microbeads, wherein a proportion of the polynucleotides associated with each bead are able to capture analyte, as in conventional methods. According to the inventors' approach, however, a proportion of the polynucleotides include a hairpin sequence capable of self-dimerisation. The method also relies on conventional barcoding, in which all of the analyte-capturing polynucleotides associated with a single bead have the same barcode sequence to each other, but a different barcode sequence from the analyte-capturing polynucleotides associated with other beads used in the same experiment. According to the inventors' approach, both the analyte capture polynucleotides and the hairpin polynucleotides associated with the same bead have the same identifying barcode sequence. In conventional methods, the barcode sequence is used to identify captured analytes from the same sample that are co-compartmentalised with a bead for analyte capture and barcoding. However, analytes from the same sample can be wrongly identified as being from separate samples if the sample was co-compartmentalised during the experiment/library preparation with more than one bead. To avoid this, a relatively low ratio of beads:sample are conventionally used, resulting in considerable loss of sample and limiting the number of samples that can be analysed per experiment. The invention solves this problem by allowing sample contact with multiple beads to be detected directly and computationally resolved, so that sample data is not lost. Hence, a higher ratio of beads:sample can be used, reducing lost sample and increasing the number of samples that can be analysed per experiment.

According to the invention, sample analytes are co-compartmentalised with beads under conditions that allow for multiple beads to be co-compartmentalised with single or a small number of samples, or the analytes thereof. The compartments are incubated under conditions that allow for capture of analytes in the sample by the analyte capture polynucleotides of co-compartmentalised beads, and under conditions that allow for dimerization of the hairpin sequences of polynucleotides from the same or different beads (i.e. where multiple beads are co-compartmentalised). Dimerisation of hairpin polynucleotides from different beads can be detected because such dimers include two different barcode sequences. Further, the sample analytes that were co-compartmentalised with multiple beads can be identified because they share a barcode sequence with the dimers.

Accordingly, in a first aspect, the invention provides a micro-particle comprising a micro-bead and an array of polynucleotides, wherein each polynucleotide of the array comprises, in a 5′ to 3′ direction: (a) a PCR handle sequence; (b) a barcode sequence (BC), wherein the barcode sequence of each polynucleotide in the array is the same as the barcode sequence of essentially each other polynucleotide in the array; and (c) an analyte capture region on a proportion of the polynucleotides and a hairpin sequence on a proportion of the polynucleotides. The polynucleotides are typically conjugated to the surface of the micro-bead at the 5′ end of the polynucleotide. However, hydrogel-type beads, where the polynucleotides are embedded in the microbead, may also be used. The analyte capture region is typically at the 3′ end (terminus) of the polynucleotides. The hairpin sequence is typically at the 3′ end (terminus) of the polynucleotides. The polynucleotides may therefore be conjugated to the surface of the micro-bead at the 5′ end of the polynucleotide and the hairpin may be at the 3′ end (terminus) of the polynucleotides. The polynucleotides also typically include a “unique” molecular identifier (UMI) sequence. The UMI is also located between the PCR handle sequence and the analyte capture region/hairpin sequence and 3′ or 5′ to the barcode sequence. The polynucleotide of the array may further comprise a cleavable-linker proximal to the bead/between the 5′ end conjugated to the micro-bead and the PCR handle sequence. Each polynucleotide of the array typically comprises a single barcode sequence, i.e. a continuous identifier sequence that is shared by essentially all of the analyte capture polynucleotides and hairpin polynucleotides of the same micro-particle/associated with the same single micro-bead. Each polynucleotide of the array typically does not comprise both an analyte capture region and a hairpin sequence, i.e. each of polynucleotides of the array comprises an analyte capture region or a hairpin sequence exclusively.

In a further aspect the invention provides a plurality of micro-particles, wherein each micro-particle comprises a micro-bead and an array of polynucleotides, wherein each polynucleotide of the array comprises, in a 5′ to 3′ direction: (a) a PCR handle sequence; (b) a barcode sequence; and (c) an analyte capture region on a proportion of the polynucleotides and a hairpin sequence on a proportion of the polynucleotides; and wherein each barcode sequence of the polynucleotide array of each micro-particle is different to each barcode sequence of the polynucleotide array of each other micro-particle. Each of the plurality of micro-particles may be a micro-particle of the invention as described above and herein.

In a further aspect the invention provides a kit comprising (i) a plurality of polynucleotides, each comprising a first ligation linker and an analyte capture region; and (ii) a plurality of polynucleotides each comprising the first ligation linker and a hairpin sequence. The kit may further comprise a plurality of micro-particles, wherein each micro-particle comprises a micro-bead and an array of polynucleotides, wherein each polynucleotide comprises, in a 5′ to 3′ direction: (a) a PCR handle sequence; (b) a barcode sequence, wherein each barcode sequence of the polynucleotide array of each micro-particle is different to each barcode sequence of the polynucleotide array of each other micro-particle; and (c) a second ligation linker for ligation to the first ligation linker.

In a further aspect the invention provides method for synthesising a micro-particle, the method comprising: (I) providing (i) a plurality of polynucleotides each comprising a first ligation linker and an analyte capture region; and (ii) a plurality of polynucleotides each comprising the first ligation linker and a hairpin sequence; (II) providing a micro-particle, wherein the micro-particle comprises a micro-bead and an array of polynucleotides, wherein each polynucleotide of the array comprises, in a 5′ to 3′ direction: (a) a PCR handle sequence; (b) a barcode sequence, wherein each barcode sequence of the polynucleotide array of each micro-particle is different to each barcode sequence of the polynucleotide array of each other micro-particle; and (c) a second ligation linker for ligation to the first ligation linker; and (III) contacting the micro-particle with a mixture of the plurality of polynucleotides (i) and the plurality of polynucleotides (ii) under conditions suitable for ligating the first ligation linker to the second ligation linker. The method produces a micro-particle of the invention as described above and herein. The invention further provides a micro-particle produced by the method.

In a further aspect, the invention provides a method for synthesising a plurality micro-particles according to the invention, the method comprising: (I) providing (i) a plurality of polynucleotides each comprising a first ligation linker and an analyte capture region; and (ii) a plurality of polynucleotides each comprising the first ligation linker and a hairpin sequence; (I) providing a plurality of micro-particles, wherein each micro-particle comprises a micro-bead and an array of polynucleotides, wherein each polynucleotide of an array comprises, in a 5′ to 3′ direction: (a) a PCR handle sequence; (b) a barcode sequence, wherein the barcode sequence of each polynucleotide in an array is the same as the barcode sequence of essentially each other polynucleotide in the array of a micro-particle; and wherein the array of polynucleotides of each micro-particle has a different barcode sequence from the array of polynucleotides of essentially each other micro-particle; and (c) a second ligation linker for ligation to the first ligation linker; and (III) contacting the micro-particles with a mixture of the plurality of polynucleotides (i) and the plurality of polynucleotides (ii) under conditions suitable for ligating the polynucleotides comprising the first ligation linker to polynucleotides comprising the second ligation linker. The method produces a plurality of micro-particles of the invention as described above and herein. The invention further provides a plurality of micro-particles produced by the method.

In a further aspect, the invention provides a method of generating a library of analytes from a plurality of samples, the method comprising: (I) providing a plurality of micro-particles as described above, wherein the micro-particles are divided between a plurality of compartments; and wherein the samples are divided between the same compartments, such that contact is made between co-compartmentalized micro-particles, or the arrays of polynucleotides thereof, and sample; (II) incubating the micro-particles, or the arrays of polynucleotides thereof, with the analytes of co-compartmentalized samples under conditions suitable to allow binding between the sample analytes and the analyte capture regions of the arrays of polynucleotides, such that sample analytes are captured for the library; and (III) incubating the micro-particles, or the arrays of polynucleotides thereof, under conditions suitable to allow different polynucleotides of the arrays comprising hairpin sequences to dimerise by annealing their complementary hairpin sequences; wherein (II) and (III) may be conducted in either order, or combined together. The polynucleotides may be released from the micro-beads at, or in between, any of steps (I) to (III), for example by cleaving a cleavable linker as described above, for example using UV light. Step (III) may comprise heating the compartments to a temperature suitable for denaturing the hairpin structures; and cooling the compartments to a temperature suitable for different polynucleotides comprising the hairpin sequence to dimerise. The method may further comprise filling in the single stranded overhangs of the dimerised polynucleotides to produce a set of double stranded polynucleotides comprising a hairpin sequence flanked by two barcode sequences, for example by reverse transcription using the free 5′ ends of the dimers as template to produce the double stranded polynucleotides comprising a hairpin sequence and two barcode sequences. The method may further comprise removing polynucleotides (e.g. non-dimerised polynucleotides) comprising the hairpin sequence, for example using a single-strand specific endo- or exonuclease. The method may further comprise amplifying the polynucleotides comprising a hairpin sequence and two barcode sequences. The method may further comprise separating the analyte capture polynucleotides, the analyte-bound polynucleotides, and/or the amplified products thereof, from the polynucleotides comprising a hairpin sequence and two barcode sequences. The method may further comprise sequencing the polynucleotides comprising a hairpin sequence and two barcode sequences.

The method may further comprise analyzing the sequences to determine which library analytes/analytes that were capture for the library were co-compartmentalised with single or multiple micro-particles or the polynucleotides thereof, or to determine how many micro-particles or the polynucleotides thereof were co-compartmentalised with a library analyte during production of the library. The method may comprise identifying a captured analyte from a sample that was co-compartmentalized during library generation with multiple micro-particles, or the arrays of polynucleotides thereof, the method comprising: (I) identifying a polynucleotide that was produced by the methods the invention, and that comprises the hairpin sequence and two different barcode sequences; (II) identifying an analyte that was captured by a polynucleotide that shares either of the two barcode sequences with the polynucleotide identified in step (I); and (III) identifying the captured analyte identified in step (II) as being from a sample that was co-compartmentalized during library generation with multiple micro-particles, or the arrays of polynucleotides thereof. The method may comprise identifying a captured analyte from a sample that was co-compartmentalized during library generation with two different micro-particles only, or the arrays of polynucleotides thereof, the method comprising: (I) identifying a polynucleotide that was produced by the methods of the invention, and that comprises the hairpin sequence and two different barcode sequences; (II) identifying all, or substantially or essentially all of the polynucleotide that comprise the hairpin sequence and either of the two barcode sequences of the polynucleotide identified in step (I); (III) determining that all of the polynucleotides identified in step (II) do not comprise any barcode sequence other than the two barcode sequences of the polynucleotide identified in step (I); (IV) identifying an analyte captured by a polypeptide that shares the either one of the two barcode sequences as the polynucleotides identified in steps (I) and (II); and (V) identifying the analyte identified in step (IV) as being from a sample that was co-compartmentalized during library generation with two micro-particle only, or the arrays of polynucleotides thereof. The method may comprise identifying a set of captured analytes from sample that was co-compartmentalized during library generation with multiple micro-particles, or the arrays of polynucleotides thereof, the method comprising: (I) identifying a set of polynucleotides that were produced by the methods of the invention and that each comprise the hairpin sequence and two different barcode sequences, wherein all of said barcode sequences in the set of polynucleotides share a string of different combinations of the barcode sequences between the polynucleotides of the set; (II) identifying a set of analytes captured by polynucleotides that share any of the barcode sequences with the set of polynucleotides identified in step (I); and (III) identifying the set of analytes identified in step (II) as being from sample that was co-compartmentalized during library generation with multiple micro-particles, or the arrays of polynucleotides thereof. The method may comprise identifying a captured analyte from sample that was co-compartmentalized during library generation with one micro-particle only, or the arrays of polynucleotides thereof, the method comprising: (I) identifying a polynucleotide that was produced by the methods of the invention, and that comprises the hairpin sequence and two identical barcode sequences; (II) identifying all of the polynucleotide that were produced by the methods of the invention, and that comprise the hairpin sequence and barcode sequence of the polynucleotide identified in step (I); (III) determining that all of the polynucleotides identified in step (II) comprise two copies of the same barcode sequence; (IV) identifying an analyte captured by a polypeptide that shares the same barcode sequence as the polynucleotides identified in steps (I) and (II); and (V) identifying the analyte identified in step (IV) as being from a sample that was co-compartmentalized during library generation with one micro-particle only, or the arrays of polynucleotides thereof.

The invention will now be described in more detail, by way of example and not limitation, and by reference to the accompanying drawings. Many equivalent modifications and variations will be apparent to those skilled in the art when given this disclosure. Accordingly, the exemplary embodiments of the disclosure set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended items, the singular forms “a”, “an”, and “the” include plural referents unless the content clearly dictates otherwise. Thus, for example, reference to “a polynucleotide” includes two or more such polynucleotides.

Section headings are used herein for convenience only and are not to be construed as limiting in any way.

### DESCRIPTION OF THE FIGURES

FIG. 1—Tapestation as described in Example 1.

Electrophoresis gel with arrows indicating the expected band sizes for hairpin monomers and dimers. Hairpin monomers can form stable dimers after extension of the 5′-overhangs by a reverse transcriptase (Maxima H minus, lane 6), a thermostable DNA polymerase (Kappa HiFi, lane 5) and a mesophilic DNA polymerase (phi29, lane 7). Dimerization was greatest with reverse transcriptase, but extensive free monomers are observed in all conditions.

FIG. 2—Tapestation as described in Example 2.

Electrophoresis gel with arrows indicating the expected band sizes for hairpin monomers and dimers. Treatment with single-strand specific nucleases including a 5′->3′ exonuclease (RecJf, lane 4), an endonuclease (mung bean nuclease, lane 5) and a 3′->5′ exonuclease (Exonuclease I) all significantly reduced free hairpin. RecJf had minimal effect on the dimer band.

FIG. 3—Tapestation as described in Example 3.

Left: Electrophoresis gel demonstrating detection of homo- and hetero-dimers after RT using two oligos of different sizes (A & B) and hairpin lengths of 12 (Lane 2) or 20 (lane 3). Upper right: Electropherogram of lane 2 showing formation of homo- and hetero-dimers of two oligos of different sizes (A & B) and hairpin length of 12. Lower right: Electropherogram of lane 3 showing formation of homo- and hetero-dimers of two oligos of different sizes (A & B) and hairpin length of 20. RecJf treatment has effectively removed hairpin monomers from both reactions.

FIG. 4—Three exemplary schemes (A, B & C) for making bead-hashing beads.

Scheme A comprises a BC and a UMI on the bead-attached polynucleotide prior to ligation of the 3′ capture sequence or the 3′ hairpin sequence. Schemes B and C involve a BC on the bead-attached polynucleotide and the UMI is provided on the 3′ capture sequence. Linker is typically hexaethylene glycol, an 18 atom spacer, but could be a non-nucleotide spacer of other lengths or a nucleotide spacer of ˜3 or more bases; PC is a photocleavable linker; PCR1 is a PCR handle of known sequence long enough for optimal binding of a PCR primer (typically, “AAGCAGTGGTATCAACGCAGAGTAC” or “TACACGACGCTCTTCCGATCT”); BC is the barcode region, unique to each bead; S is an optional spacer region of 3 or more known bases, used to better define boundaries between barcode and UMI and UMI and capture sequence; UMI is the unique molecular identifier; L is the ligation site; Capture is the analyte capture sequence; Palindrome is a palindromic sequence, that spontaneously forms a hairpin structure; B is an optional biotinylation (or desthiobitoninylated) nucleotide(s) to assist in downstream separation of bead-hashing libraries.

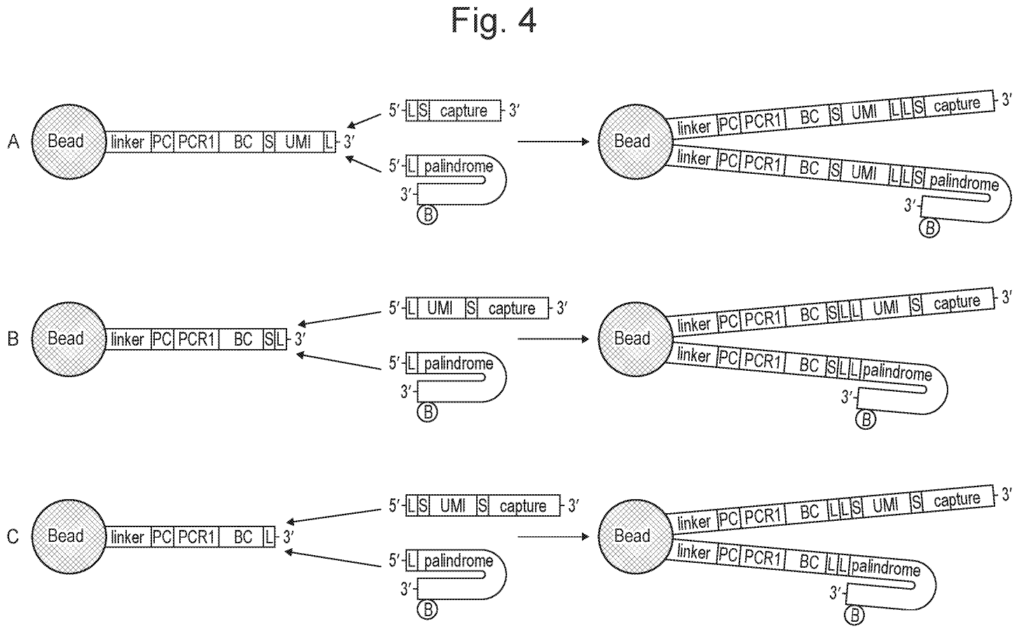

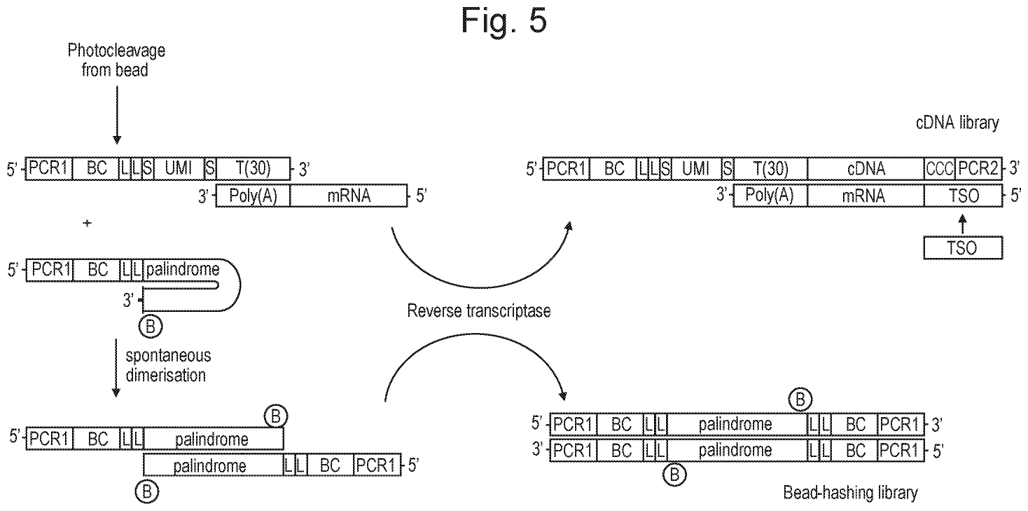

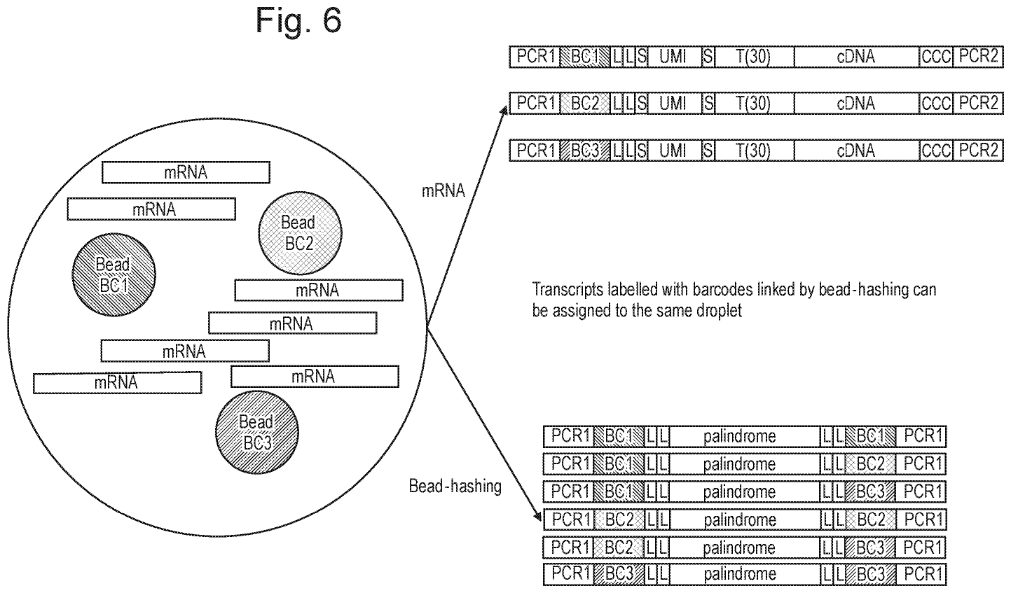

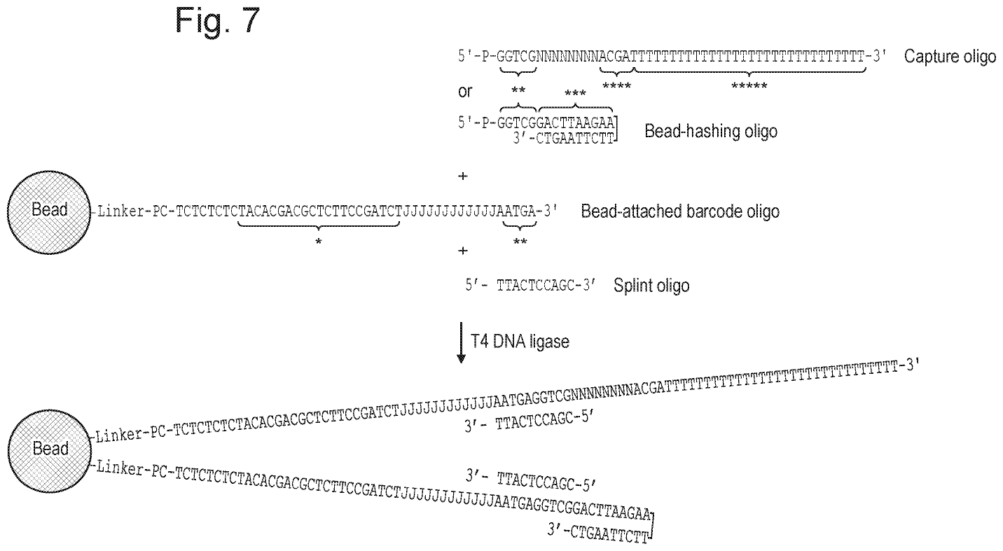

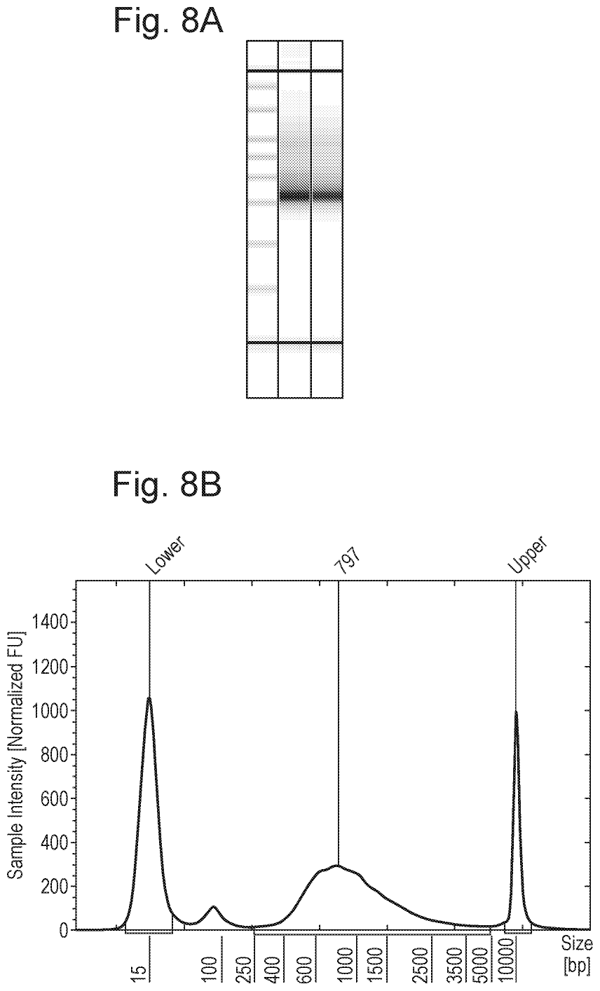

FIG. 5—A schematic of cDNA generation and dimerised palindrome extension using reverse transcription, involving the bead of FIG. 4, scheme C. TSO is a template switch oligonucleotide. PCR2 is a PCR handle of known sequence.

FIG. 6—Schematic showing the generation of polynucleotides comprising cDNA and polynucleotides comprising the palindromic sequences from a droplet comprising multiple beads and mRNA.

FIG. 7—Exemplary method of enzymatic ligation of an analyte capture sequence or palindromic sequence to a bead-attached polynucleotide, using T4 DNA ligase and a splint oligonucleotide. * is a PCR handle sequence. ** are sequences that are complementary to the splint oligonucleotide. *** is a palindromic hairpin sequence. **** is a spacer sequence. ***** is an analyte capture sequence. J is a nucleotide of the barcode sequence. N is a nucleotide of the UMI sequence.

FIG. 8—A. Electrophoresis gel showing production of polynucleotides comprising an analyte capture sequence following ligation to a bead attached oligonucleotide following the enzymatic ligation as shown in FIG. 7. Lane 1 shows a DNA ladder. Lanes 2 and 3 show a single band of a size corresponding to a single polynucleotide comprising both bead-attached and analyte-capture portions. B. Showing cDNA generated using beads prepared by enzymatic ligation, according to the method disclosed in Example 4.

### DETAILED DESCRIPTION OF THE INVENTION

### Micro-Particles

In some embodiments the invention relates to micro-particles. The micro-particles of the invention comprise a micro-bead and an array of polynucleotides as described herein. Microbeads are typically less than 500 μm, or less than 400 μm, 300 μm or 200 μm in diameter, for example, between about 1 and 500 μm, 1 and 200 μm, 5 and 100 μm, 5 and 50 μm, or 10 and 40 μm. Most typically the microbeads are about 1 to 50 μm, but small microbeads of about 1 to 15 μm or 8 to 20 μm are particularly advantageous for use with the invention. Typically, a micro-bead is approximately spherical or sphere-like. Micro-beads with surface-attached polynucleotides are well-known in the art and may be made from, for example, a biocompatible polymer such as polystyrene, polyacrylamide or hydroxylated methacrylic polymer, or from controlled pore glass.

The array of polynucleotides typically comprises at least 200, or at least 1000, 10,000, 100,000, 1,000,000, 10⁷, 10⁸, 10⁹ or 10¹⁰ polynucleotides, for example between 200 and 10¹² or between 50, 200, 10², 10³, 10⁴ or 10⁵ and 10¹¹, or between 10⁶, 10⁷, 10⁸, or 10⁹ and 10¹⁰ polynucleotides, or most typically between 10⁶ and 10¹⁰ polynucleotides. The micro-particles may be of a conventional type in which the polynucleotides of the array are attached or conjugated at one end to the surface of the micro-bead. The other end of each polynucleotide is typically free in solution. Typically, it is the 5′ end of the polypeptide that is conjugated to the surface of the micro-bead and the 3′ end that that is free in solution. In this case the polynucleotides are typically synthesised or assembled on the bead in a 5′ to 3′ direction, using methods known in the art. However, the reverse is also contemplated, for example as described in PCT/GB2021/051151.

Dissolvable beads and hydrogel beads have also been described and are encompassed in the present disclosure. Dissolvable beads may, for example, be made from crosslinked acrylamide with disulfide bridges that are cleaved with dithiothreitol. An array of polynucleotides may be embedded in the bead matrix and released when the bead is dissolved. Hence, a micro-particle comprising a micro-bead and an array of polynucleotides bound to the micro-bead as described herein may be a typical micro-bead with surface bound polynucleotide or a dissolvable bead with embedded polynucleotides.

Polynucleotides may be synthesised on the bead using methods known in the art. For example, the phosphoramidite method may be used, in which one nucleotide is added per synthesis cycle. Identifier sequences, such as a barcode sequence and UMI sequence are typically added, for example using degenerative synthesis or split-and-pool synthesis. Longer sequence elements may in some cases be added using enzymatic ligation methods, such as using DNA ligase, chemical ligation methods, such as phosphoramidate ligation, and/or click chemistry ligation methods, such as the azide-alkyne cycloaddition reaction. Suitable methods are known in the art.

Polynucleotides may be linked to the bead by any means known to the skilled person, for example via hexaethylene glycol, an 18 atom spacer, a non-nucleotide spacer of other lengths, or a nucleotide spacer of 3 or more bases.

The invention may relate to a plurality of micro-particles as described herein. A typical number of micro-particles is sufficient to conduct a typical experiment or to generate one or more libraries. For example, the plurality of micro-particles may comprise at least 1000, or at least about 10,000, or 100,000, or 500,000, for example between about 1000 and 2 million, or between 50,000 and 1.5 million, or most typically between about 100,000 and 1.5 million micro-particles. The number of micro-particles may be higher than used in typical experiments to date, because the invention allows higher loading of micro-particles, e.g. to achieve a minimum of essentially 1 micro-particle per droplet, and thus maximise the number of samples that can be analysed. Typically, the barcode sequence of each polynucleotide in an array is the same as the barcode sequence of essentially each other polynucleotide in the array of a micro-particle; and the array of polynucleotides of each micro-particle has a different barcode sequence from the array of polynucleotides of essentially each other micro-particle. However, it is sufficient that each barcode sequence of the polynucleotide array of each micro-particle is different to each barcode sequence of the polynucleotide array of each other micro-particle. The micro-particles of the plurality of micro-particles may have any of the features described herein for single micro-particles.

The set of polynucleotides associated with each micro-particle have a different shared barcode sequence/set of barcode sequences from the polynucleotides associated with essentially each of the other micro-particles. Hence, the analytes that are captured by the set of polynucleotides associated with each micro-particle can be distinguished. The potential diversity of barcode sequences is dependent on the barcode sequence length and composition, as described elsewhere herein. The barcode sequence will typically be long enough that the potential sequence diversity is well in excess of the plurality of micro-particles, typically at least 50× or 100× in excess. For example, for a typical experiment using a pool of approximately 150,000 micro-particles, a 12-nucleotide barcode sequence provides a potential diversity of 4¹² (16,777,216) different sequences, which provides an excess of barcode sequences to beads of >100×.

### Polynucleotides

The terms “polynucleotide”, “oligonucleotide” or “oligo” may in some cases be used herein interchangeably, and refer to a string of nucleotide monomers in a chain typically linked by phosphodiester bonds. As used herein, a polynucleotide may be a chain of nucleotides of any length, whilst an oligonucleotide typically comprises up to 50 nucleotides. In some cases, the polynucleotides of the invention may be at least 50, or at least 56, 60, 70, 80, 90, 100, 110, 120 or 125 and/or up to 130, 140, 160, 180, 200, 225, 250, 275, 300, 350, 400, 500 nucleotides or more in length, for example between 50 and 500 or 400 or 300 or 200 nucleotides in length, or most typically 64 to 122 in length. For example, a typical hairpin polynucleotide as described herein and including BC and UMI sequences made up of monomer units may have a typical length of about 64 base pairs, or more typically about 72 base pairs. A typical analyte capture polynucleotide as described herein and including BC and UMI sequences made up of monomer units may have a typical length of about 82 bp. Similar hairpin and analytes capture polynucleotides comprising BC and UMI sequences made up of dinucleotide units would typically be about 40 bp longer.

The polynucleotides are typically DNA (single-stranded DNA) but could also be RNA. Polynucleotides have a chemical orientation defined by the position of the linking carbon in the five-carbon sugar of each consecutive nucleotide in the chain. Polynucleotides may be manufactured by the addition of nucleotides at either the 5′ end (manufacture in a 5′ direction) or the 3′ end (manufacture in a 3′ direction) to elongate the chain. Likewise, sequence elements along the length of a polynucleotide have a sequential order defined by the directionality of the chain of nucleotides that is either 5′ to 3′ or 3′ to 5′.

In some cases, the polynucleotides comprise the following sequence elements in a 5′ to 3′ direction: (a) optional cleavable linker; (b) a PCR handle sequence; (c) a barcode sequence (BC); polynucleotides also typically comprise a “unique” molecular identifier sequence (UMI); the BC and UMI (where present) may be in either orientation; and (d) an analyte capture region and/or a hairpin sequence, as described herein.

In some cases, the polynucleotides comprising an analyte capture region comprise a UMI, and the polynucleotides in the array comprising a hairpin sequence do not comprise a UMI. In some cases, the polynucleotides comprising a hairpin sequence do not comprise an analyte capture region. For example, the polynucleotides may comprise two sub-populations of polynucleotides, wherein the polynucleotides in the first sub-population comprise the following sequence elements in a 5′ to 3′ direction: (a) optional cleavable linker; (b) a PCR handle sequence; (c) a barcode sequence (BC); (d) a “unique” molecular identifier sequence (UMI); and (e) an analyte capture region, as described herein, and wherein the polynucleotides in the second sub-population comprise the following sequence elements in a 5′ to 3′ direction: (a) optional cleavable linker; (b) a PCR handle sequence; (c) a barcode sequence (BC); (d) a hairpin sequence, as described herein. Other examples are shown in FIG. 4.

Typically, the PCR handle sequence and/or any other sequence elements 5′ of the hairpin sequence and/or analyte capture region are also the same for each polynucleotide of the micro-particle. In some cases, all of the polynucleotides also have the same analyte capture region. In these embodiments, the polynucleotides of a micro-particle only differ in that some include a further 3′ end hairpin sequence and optional loop, whilst others have the analyte capture region at the 3′ end. This simplifies manufacture of the polynucleotides, since only the 3′ end elements, which are added to the polynucleotides last when manufactured in a typical 5′ to 3′ direction, differ amongst the polynucleotides of a single micro-particle.

In some cases, the polynucleotides may comprise non-nucleotide linking elements (i.e. spacers), for example phosphoramidite spacers, such as 17-O-(4,4′-Dimethoxytrityl)-hexaethyleneglycol, 1-[(2-cyanoethyl)-(N,N-diisopropyl)]-phosphoramidite (HEG). For example, a spacer may be included 3′ to the 3′ analyte capture region, or between the 3′ analyte capture region and the bead. The polynucleotides may also comprise further sequence elements in addition to those defined herein. However, the analyte capture region or the hairpin sequence is typically at the 3′ terminus of the polynucleotide.

In some cases, the polynucleotides may comprise a spacer 5′ and/or 3′ of the barcode sequence. In some cases, the polynucleotides may comprise a spacer 5′ and/or 3′ of the UMI sequence. In some cases, the polynucleotides may comprise a spacer 3′ of the analyte capture region. Spacers may be nucleotide or non-nucleotide elements. Typically, the spacer is a nucleotide sequence. The nucleotide spacer typically comprises 3 or more known bases. The nucleotide spacer may comprise 3 to 20 known bases, such as 3 to 10 known bases, 3 to 5 known bases, or 3, 4 or 5 known bases. The nucleotide spacer may comprise or consist of one or more nucleotide blocks as described herein. Spacers can be used to better define the boundaries between the PCR handle and the barcode sequence, the barcode sequence and the UMI, and/or the UMI and the capture sequence. The addition of short constant spacer sequences improves downstream analysis of sequencing data that it ultimately generated. The spacer may comprise a ligation linker as described herein, in particular the nucleotide ligation linkers. In some cases, the polynucleotides comprise the following sequence elements in a 5′ to 3′ direction: (a) optional cleavable linker; (b) a PCR handle sequence; (c) optional spacer; (d) a barcode sequence; (e) optional spacer; (f) a UMI sequence; (g) optional spacer; and (h) an analyte capture region and/or a hairpin sequence, as described herein. In some cases, the polynucleotides comprise the following sequence elements in a 5′ to 3′ direction: (a) optional cleavable linker; (b) a PCR handle sequence; (c) optional spacer; (d) a UMI sequence; (e) optional spacer; (f) a barcode sequence; (g) optional spacer; and (h) an analyte capture region and/or a hairpin sequence, as described herein. In some cases, the polynucleotides comprise the following sequence elements in a 5′ to 3′ direction: (a) optional cleavable linker; (b) a PCR handle sequence; (c) optional spacer; (d) a barcode sequence; (e) optional spacer; and (f) an analyte capture region and/or a hairpin sequence, as described herein. As described above, in some cases the polynucleotides comprising an analyte capture region comprise a UMI, and the polynucleotides in the array comprising a hairpin sequence do not comprise a UMI. In some cases, the polynucleotides comprising a hairpin sequence do not comprise an analyte capture region. For example, the polynucleotides may comprise two sub-populations of polynucleotides, wherein the polynucleotides in the first sub-population comprise the following sequence elements in a 5′ to 3′ direction: (a) optional cleavable linker; (b) a PCR handle sequence; (c) optional spacer; (d) a barcode sequence; (e) optional spacer; (f) a UMI sequence; (g) optional spacer and (h) an analyte capture region, as described herein, and wherein the polynucleotides in the second sub-population comprise the following sequence elements in a 5′ to 3′ direction: (a) optional cleavable linker; (b) a PCR handle sequence; (c) optional spacer; (d) a barcode sequence; (e) optional spacer; and (f) a hairpin sequence, as described herein.

In some cases, a spacer is present between the UMI and the analyte capture region but is not present between the UMI and the hairpin sequence. In some cases, a spacer is present between the BC and the analyte capture region but is not present between the BC and the hairpin sequence.

The polynucleotides of the present invention may in some cases comprise any other suitable feature described in PCT Publication No. WO 2021/229230.

Polynucleotides may comprise any combination of natural or canonical nucleotides (i.e., “naturally occurring” or “natural” nucleotides), which include adenosine, guanosine, cytidine, thymidine and uridine. The polynucleotides may also comprise nucleotide analogues. For example, the polynucleotide may include one or more peptide nucleotides, in which the phosphate linkage found in DNA and RNA is replaced by a peptide-like N-(2-aminoethyl)glycine. Peptide nucleotides undergo normal Watson-Crick base pairing and hybridize to complementary DNA/RNA with higher affinity and specificity and lower salt-dependency than normal DNA/RNA oligonucleotides and may have increased stability. The polynucleotide may include one or more locked nucleotides (LNA), which comprise a 2′-O-4′-C-methylene bridge and are conformationally restricted. LNA form stable hybrid duplexes with DNA and RNA with increased stability and higher hybrid duplex melting temperatures. The polynucleotide may include one or more Propynyl dU (also known as pdU-CE Phosphoramidite, or 5′-Dimethoxytrityl-5-(1-Propynyl)-2′-deoxyUridine,3′-[(2-cyanoethyl)-(N,N-diisopropyl)]-phosphoramidite). The polynucleotide may include one or more unlocked nucleotides (UNA), which are analogues of ribonucleotides in which the C2′-C3′ bond has been cleaved. UNA form hybrid duplexes with DNA and RNA, but with decreased stability and lower hybrid duplex melting temperatures. LNA and UNA may therefore be used to finely adjust the thermodynamic properties the polynucleotides in which they are incorporated. The polynucleotide may include one or more triazole-linked DNA oligonucleotides, in which one or more of the natural phosphate backbone linkages are replaced with triazole linkages, particularly when click chemistry is used for synthesising the polynucleotide. The polynucleotide may include one or more 2′-O-methoxy-ethyl bases (2′-MOE), such as 2-Methoxyethoxy A, 2-Methoxyethoxy MeC, 2-Methoxyethoxy G and/or 2-Methoxyethoxy T. The polynucleotide may include one or more 2′-O-Methyl RNA bases. The polynucleotide may include one or more 2′-fluoro bases, such as fluoro C, fluoro U, fluoro A, and/or fluoro G. Other specific examples of nucleotide analogues include 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, 5-Methyl dC, 5-Nitroindole, 5-hydroxybutynl-2′-deoxyuridine (Super T) and 8-aza-7-deazaguanosine (Super G). In some cases, the polynucleotide may include super T 2,6-Diaminopurine (2-Amino-dA) and/or 5-Methyl dC. The polynucleotide may include one or more biotinylated nucleotides. In some cases, the polynucleotide comprises at least two, or at least 3, 4, 5, 6, 7, 8, 9, 10, 12, 14, 16, 18, 20, 25, 30, 35, 40, or up to 2%, 5%, 10%, 15%, 20%, 25%, 30%, 35%, 40% or more nucleotide analogues and/or biotinylated nucleotides, or any one type of nucleotide analogue as described herein.

### Analyte Capture Polynucleotides/Regions

The polynucleotides associated with a micro-particle as described herein include “analyte capture polynucleotides”, i.e. polynucleotides comprising an “analyte capture region”.

An analyte capture region may be any nucleotide sequence suitable for capturing analyte in a sample. In some cases, the analyte(s) may be biological analytes or may be selected from polynucleotides DNA and/or RNA, or from oligonucleotides, DNA, cDNA, RNA, mRNA, rRNA, tRNA, snRNA, siRNA and/or ribozymes, proteins, polypeptides and/or peptides, cell surface receptors or cells. Most typically the analytes are mRNA. Other examples of analytes that may be captured by suitable analyte capture regions include amino acids, metal ions, inorganic salts, polymers, nucleotides, oligonucleotides, polynucleotides, dyes, bleaches, pharmaceuticals, diagnostic agents, recreational drugs, explosives and/or environmental pollutants. Such analytes may be captured, for example, by an aptamer or other types of analyte capture region as are known in the art,

Typically, the analyte capture region sequence is at least 10, or at least 15, 20, 25 or 30 nucleotides in length, such as from about 15 to about 50, from about 20 to about 40 or from about 25 to about 35 nucleotides. Most typically the analyte capture region is 20 to 40 nucleotides in length. In some cases, the analyte capture region may comprise one or more nucleotide analogues, such as analogues described herein, that form double-stranded hybrids with higher stability than natural nucleotides. In this case, the analyte capture region could be shorter, such as at least 3, 4, 5, 6, 8 or 9, for example between 3 and 50, or 40 or 30 or 20 nucleotides in length, provided that the analyte capture region is capable of binding or hybridizing to target analyte, e.g. such that analyte sequence can be amplified as described herein. An analyte capture region may include nucleotide analogues as described herein.

In some cases, an analyte capture region may be a DNA capture region, an RNA or mRNA capture region, or a polypeptide capture region.

In one example, an analyte capture region, particularly a 3′ analyte capture region, may be a polythymidine. Polythymidine may hybridise to and capture any polynucleotide in the sample that comprises a suitable polyadenosine, such as polyadenylated mRNA. Typically, the polythymidine is at least 10, or at least 15, 20, 25 or 30 thymidines in length, such as from about 15 to about 50, from about 25 to about 35 or most typically from about 20 to about 40 thymidines. The analyte capture region may be (about) 30 thymidines in length.

In other cases, the analyte capture region(s) may comprise or consist of an aptamer. Aptamers can be produced using SELEX (Stoltenburg, R. et al., (2007), Biomolecular Engineering 24, p381-403; Tuerk, C. et al., Science 249, p505-510; Bock, L. C. et al., (1992), Nature 355, p564-566) or NON-SELEX (Berezovski, M. et al. (2006), Journal of the American Chemical Society 128, p1410-1411). Typically, an aptamer may be at least 15 nucleotides in length, such as from about 15 to about 50, from about 20 to about 40 or from about 25 to about 30 or nucleotides in length. An aptamer may bind to analyte such as small molecules, proteins, nucleic acids or cells. Aptamers may be designed or selected to bind to pre-determined target analyte(s). In one example, the aptamer may bind to a Coronaviridae protein or SARS-CoV-2 protein, as described in described in PCT Publication No. WO 2021/229230.

In some cases, an analyte capture region may comprise or consist of a biotinylated nucleotide sequence. Nucleotides or polynucleotides may be biotinylated using methods known in the art. Typically, the biotinylated sequence may be at least 10, or at least 15, 20, 25 or 30 nucleotides in length, such as from about 15 to about 50, from about 20 to about 40 or from about 25 to about 35 nucleotides. A biotinylated capture region may be used to capture any suitable target analyte comprising streptavidin or avidin.

In some cases, an analyte capture region may comprise or consist of a nucleotide sequence designed to hybridise to a complementary sequence in a target polynucleotide/analyte. In some cases, the capture region is for capturing/hybridising to transposed DNA. In this case an analyte capture region may comprise or consist of a sequence that is complementary to transposed DNA in a sample, for example to a transposed MEDS DNA sequence. In other cases, the sequence may be gene or transcript-specific, such as a polynucleotide sequence that is complementary to, or at least 80%, 85%, 90%, 95%, 98% or 99% complementary to, a viral sequence, a bacterial sequence or a sequence associated with a disease or disorder, such as a sequence from a cancer-associated antigen or a neoantigen. In some cases, the analyte capture region(s) may hybridise to a nucleotide sequence that encodes a part of a Coronaviridae protein or SARS-CoV-2 protein, as described in described in PCT Publication No. WO 2021/229230.

In other cases, the sequence may be designed to capture a polynucleotide tag added to analyte of interest.

In some cases, all of the polynucleotides, or all of the analyte capture polynucleotides, associated with a micro-particle, or with an array or plurality of micro-particles as described herein, may have the same analyte capture region. In other cases, a combination of different capture regions may be used.

### Hairpins

The polynucleotides associated with a micro-particle as described herein further include polynucleotides having a (terminal) hairpin sequence.

A hairpin sequence includes palindromic elements that will anneal together to form a hairpin structure, as is well understood in the art. The hairpin structure may optionally include additional nucleotides in between the pair of palindromic (reverse complementary) elements, e.g. a loop sequence. If the hairpin structure is melted/denatured, then two different polynucleotides having the same hairpin sequence can anneal to each other to form a dimer. Dimers form preferentially and at a higher temperature than hairpins having the same sequence because the dimers have twice as many base pairs. The hairpin length and sequence can be selected by those in the art to have a desired melting temperature for the hairpin structure and/or corresponding dimers. For example, the hairpin sequence could be designed to have an unstable hairpin structure, but be stable as a dimer, at room temperature (about 20° C., or about 18 to 23° C.).

A typical hairpin sequence may contain palindromic sequence elements, each about 5, or about 6, 7, 8, 9, 10, 12, or 15 to about 30, or about 27, 25, 20, or 18 nucleotides in length, with an optional loop sequence element or spacer in between the two palindromic regions. Most typically, each of the pair of palindromic sequences is about 6 to 20 nucleotides in length. In some cases, the palindromic regions may comprise one or more nucleotide analogues, such as analogues described herein, that form double-stranded hybrids with higher stability than natural nucleotides. In this case, the palindromic regions could be shorter, such as at least 3, 4, or 5, nucleotides in length, provided that the hairpin sequence is able to form both a hairpin structure and dimers.

In some cases, the hairpin sequence may contain sequence elements that are substantially palindromic. The term substantially palindromic as used herein is intended to refer to sequence elements that comprise a degree of complementarity sufficient to form a stable hairpin structure. The stability of the hairpin may be assessed, for example, at 20° C., 25° C., 30° C. or 37° C. The hairpin may be considered stable if for example, less than 10%, less than 5%, less than 1% or less than 0.1% of hairpin sequences are linear in solution under the conditions tested. A hairpin sequence may therefore contain first and second sequence elements, wherein the second sequence element comprises (a) a complementary sequence to the sequence of the first sequence element, or (b) comprises a sequence having one, two, three, four or five nucleotide substitutions to the sequence of (a).

Typically, the hairpin sequence/palindromic region spontaneously forms a hairpin structure.

In some cases, no loop sequence per se is included, although a small number of bases at the centre of a palindromic sequence may spontaneously form a non-base pairing loop. Not including a non-palindromic loop sequence increases the number of paired bases when two “hairpin” polynucleotides form a dimer. When a non-palindromic loop sequence is present, it is typically less than about 50, or less than about 40, 35, 30, 25, 20, 15 or 10 nucleotides in length and is typically about 4 to 12, or 6 to 10 nucleotides in length. The loop may typically have a simple structure with no base-pairing between nucleotides of the loop. However, sequences in between the palindromic elements that provide additional secondary structure are also contemplated, for example a clover structure. Such structures fall within the term “hairpin” as used herein, provided the sequences are also able to dimerise, as described herein.

The micro-particles described herein and used in the invention comprise an array of polynucleotides, wherein a proportion of the polynucleotides are analyte capture polynucleotides, as described herein, and a proportion of the polynucleotides have a hairpin sequence. Typically, at least 10% of the polynucleotides include an analyte capture region. Typically, at least 0.0000001% of the polynucleotides include the hairpin sequence/structure. Typically, all, or essentially all of the polynucleotides associated with the micro-particle have either an analyte capture region or a hairpin sequence/structure. In some cases, all of the polynucleotides of the array comprise an analyte capture region and a proportion of the polynucleotides of the array further comprise a hairpin sequence/structure that is 3′ to the analyte capture region/3′ terminal. For example, up to 80%, or up to 70%, 60%, 50%, 40%, 30%, 20%, 10%, 1% or 0.1% of the array may include the hairpin. In other cases, all or essentially all of the polynucleotides of the array comprise either an analyte capture region or a hairpin sequence/structure, but not both. The ratio of array polynucleotide having an analyte capture region to array polynucleotides having a hairpin sequence/structure may be, for example, between 1000000:1 to 1:100, or 100000:1 to 1:100, or 10000:1 to 1:100, or 100:1 to 1:100, or 10:1 to 1:100; or 1000000:1 to 1:10, or 100000:1 to 1:10, or 10000:1 to 1:10, or 1000:1 to 1:10, or 100:1 to 1:10; 1000000:1 to 1:1, or 100000:1 to 1:1, or 10000:1 to 1:1, or 100:1 to 1:1, or 10:1 to 1:1. Hence, in some embodiments a majority of the polynucleotides have an analyte capture region and a minority of the polynucleotides have a hairpin. In other embodiments, a majority of the polynucleotides have the hairpin and a minority have an analyte capture region.

In some cases, all or essentially all of the polynucleotides of the array exclusively comprise either an analyte capture region or a hairpin sequence/structure, i.e. each of the polynucleotides of the array do not comprise both an analyte capture region and a hairpin sequence. For example, a proportion of the polynucleotides in the array comprise the analyte capture region but not the hairpin sequence, and a proportion of the polynucleotides in the array comprise the hairpin sequence but not the analyte capture region. In some cases, fewer than 1% of the polynucleotides of the array comprise the analyte capture region and the hairpin sequence, such as fewer than 0.1%, fewer than 0.001%, fewer than 0.00001% or none of the polynucleotides of the array. In this way, the polynucleotides of the array comprising a hairpin sequence and the polynucleotides of the array comprising an analyte capture region comprise an identical barcode sequence allowing to the sets of polynucleotides to be identified as originating from the same array directly. By minimising the length of the polynucleotides of the array, this also reduces the chances of unintended secondary polynucleotide structure from forming, when compared to longer polynucleotides.

Typically, all of the hairpin sequences of the array of polynucleotides are the same. However, a number of different hairpin sequences could be used within each array/micro-particle, as long as the same, or essentially the same, set of hairpin sequences was used for all other micro-particles used in an experiment/method of generating a library as described herein.

Hence, in embodiments relating to a plurality of micro-particles, the hairpin sequence or set of hairpin sequences of each micro-particle is the same as the hairpin sequence or set of hairpin sequences of each other micro-particle.

In some cases, the hairpin includes a tag element that allows the hairpins/dimers (library) to be separated from the analyte capture polynucleotides/library during downstream processing as described herein. In some cases, the hairpin polynucleotides may include a biotin. For example, biotin dT may be used during synthesis of the hairpin polynucleotides.

### Identifier Sequences/Barcode Sequences

The invention uses identifier sequences. The term “identifier sequence” as used herein refers to a polynucleotide sequence tag or index that can be used to distinguish different polynucleotides or groups of polynucleotides, either alone or combination with other identifier sequences or other sequence elements. Identifier sequences are typically added to or included in a polynucleotide to capture information about the polynucleotide or an analyte associated with or captured by the polynucleotide, or the amplification products thereof. For example, an identifier sequence could indicate the source of a polynucleotide/analyte, for example a particular sample (e.g. a cell, or cell nucleus), or a spatial position, or can be used to count the number of original analytes after downstream amplification. Often a benefit of using an identifier sequence is that it allows different samples, molecules or analytes to be mixed together physically after being tagged with the identifier sequence, for more efficient downstream processing, such as amplification and sequencing. The identifier sequence is used later to distinguish different molecules, their source, or copies thereof.

In the present invention, both the analyte capture polynucleotides and the hairpin polynucleotides associated with a micro-particle include an identifier sequence, as described herein. The identifier sequence is copied when the polynucleotides are amplified, so that amplification products are also tagged by the identifier sequence. An analyte that is captured by an analyte capture polynucleotide may also be tagged by the identifier sequence of the polynucleotide that captures the analyte, for example when the captured analyte is amplified together with the analyte capture polynucleotide, including the identifier sequence.

The invention makes use in particular of a type of identifier sequence referred to herein as a barcode sequence (BC). Barcode sequences are well understood in the art and are typically used to tag/index multiple analytes from a shared source. The present invention initially, or partly, uses barcode sequences in the conventional way during sample analyte library generation. Micro-particles include an array of analyte capture polynucleotides that are used to capture sample analyte to generate the library. Each of the capture polynucleotides typically comprises the same barcode sequence. A micro-particle, and its associated array of analyte capture polynucleotides, may be conveniently compartmentalised together with one or more discrete samples (for example single cells). Analytes from the sample are subsequently captured by the analyte capture polynucleotides and tagged with the barcode. Hence, in cases where single samples (e.g. single cells) are co-compartmentalised with single micro-particle s, or the capture polynucleotides thereof, analytes from the same sample can be identified by the shared barcode sequence of the polynucleotide that captured the analyte.

Typically, each polynucleotide in the arrays disclosed herein comprise a single barcode sequence (i.e. does not comprise more than one barcode sequence). The barcode sequence is present between the PCR handle and the analyte capture region and/or hairpin sequence, i.e. to ensure that it is amplified during PCR and thus detectable. Typically, each polynucleotide in the arrays disclosed herein comprise a single copy of the single barcode sequence.

The invention addresses the problem of distinguishing between (i) a set of analytes that are all from the same sample/compartment and that were all captured by analyte capture polynucleotides of the same array/from the same micro-particle, and (ii) a set of analytes that are all from the same sample/compartment, but that were captured by the analyte capture polynucleotides of more than one array/micro-particle. Situation (ii) typically occurs when more than one micro-particle is divided into a single compartment, such that multiple arrays are contacted with the same sample.

Identifier sequences have conventionally consisted of a string of single nucleotides, generated randomly, for example using split-and-pool synthesis or degenerate polynucleotide synthesis, as described further elsewhere herein. In other cases, in accordance with the invention, the identifier sequences may have the features described in PCT/GB2021/053152, for example in the claims of that application. Instead of consisting of a string of single nucleotides, the identifier sequences comprise a series of discrete nucleotide blocks. Typically, the blocks are two or three nucleotides in length, but longer blocks can also be used. An identifier sequence could also be built up from multiple blocks of different sizes. Typically, however, each block in the same or an equivalent position in each identifier sequence is the same length. Each discrete nucleotide block has one of a pool of known sequences. An identifier sequence/BC, e.g. of an individual polypeptide, may comprise or consist of any combination of the same or different nucleotide blocks within the limitations described herein. However, the ability to differentiate between identifier sequences is improved by using a pre-determined and limited number of different nucleotide block sequences, either across the full identifier sequence or at each same or equivalent position of the identifier sequence across different polynucleotides. The sequence of each block can be compared across different polynucleotides, i.e. because they are at the same known or otherwise identifiable position in each polynucleotide. These pre-defined nucleotide block sequences may be referred to herein as a nucleotide block pool or nucleotide block sequence pool. All of the nucleotide block sequences within the pre-defined nucleotide block pool typically differ from every other nucleotide block sequence within the pool by at least two, or at least three nucleotide substitutions.

The sequence of each of the nucleotide blocks is otherwise not particularly limited unless otherwise provided herein and provided that the different nucleotide block sequences can be distinguished when sequenced.

A typical barcode sequence may include at least 3, 4, 5, 6, 7, 8, 9, 10, 11 or 12 nucleotide blocks. For example, a barcode sequence may consist of about 6 to 14, 10 to 14, or 11 to 13 nucleotide blocks where each nucleotide block pool comprises four different sequences (for example, where each nucleotide block consists of one or two natural nucleotides), or may, for example, consist of about 4 to 9, 5 to 8, or 6 to 7 nucleotide blocks where each nucleotide block pool comprises twelve different sequences (for example, where each nucleotide block consists of three natural nucleotides). The barcode sequence is typically synthesised using a number of rounds of split-and-pool synthesis corresponding to the number of nucleotide blocks in the barcode sequence.

An identifier sequence added to analytes to identify or count single molecules or single capture events, e.g. prior to an amplification step, may be referred to as a “unique molecular identifier” (UMI) sequence. UMI sequences are typically added to micro-particles such as those described herein using degenerative synthesis, as known in the art. In some cases, a different identifier sequence is added to each polynucleotide/analyte in the sample to which the identifier sequence is added. In other cases, the same identifier sequence may be added to more than one polynucleotide/analyte. For example, if both a BC sequence and a UMI sequence is to be added to each polynucleotide/analyte (e.g. at the same end), wherein the BC sequence identifies each of a number of sub-arrays of the polynucleotides/analytes, then the UMI sequence may be designed (i.e. to have sufficient total possible sequence diversity) such that essentially every polynucleotide/analyte of the same sub-array receives a different UMI sequence, but the same UMI sequence may be added to two or more polynucleotides/analytes that are in different sub-arrays. Such polynucleotides receiving the same UMI sequence can be distinguished based on their combined BC and UMI sequences. Alternatively, or in addition unique capture events can be identified based on a combination of the UMI sequence and the identity of the captured analyte. Hence, the diversity of UMIs needed depends on the experiment. However, in some cases a different UMI sequence is added to each of at least 2000, 5000, 10⁴, 10⁵, 10⁶ or 10⁷ different polynucleotides in the array of a/each micro-particle as described herein.

An identifier sequence may comprise at least 4, more typically at least 5, 6, 7 or 8 sequence units (where a sequence unit is either a single nucleotide or a nucleotide block as described herein) and up to 12, 13 or 14 sequence units or more, more typically 6 to 14, 7 to 13, 10 to 14 or 8 or 12 sequence units. 8 to 12 sequence units are most typical. The sequence units are added to, or present in, the relative polynucleotide at successive, consecutive or non-consecutive unit or block positions. The identifier sequence is defined by the sequence of the unit or block at each position. The total diversity of possible identifier sequences is determined by (i) the number of sequence units included in the identifier sequence; and (ii) the number of different units or nucleotide block sequences included in the pool of sequences that can be used at each unit/block position. If the same pool is used at every position, then the total possible diversity of sequences is equal to [the size of the pool]×[the number of sequence units or nucleotide blocks in the identifier sequence]. In some cases, the total diversity of possible identifier sequences, is at least 10, or at least 20, 50, 100, 200 or 500 times in excess of the number of sample polynucleotides. For example, 10 to 12 sequence units, with four possible options for each unit, provides about 1e+6 to 1.6e+7 possible unique identifier sequences. A typical bulk mRNAseq experiment might start with 1 μg of total RNA and contain about 4e+10 mRNA molecules. Hence, in this example, the number of mRNA molecules is greater than the number of identifier sequences in the pool, but smaller than the number of unique combinations of identifier sequences added at both ends. Moreover, the mRNA sample may be made up of many thousands of non-identical transcripts, which can further distinguish between indexed library sample polynucleotides amplified from different sample molecules flanked by an identifier sequence at each end (as described further elsewhere herein). A typical sequencing depth might be only 2e+7 to 1e+8 for a given sample. Hence, the chances of two identical sample RNA transcripts getting an identical pairing of flanking identifier sequences becomes, in this example, vanishing small.

Hence, the number of sequence units chosen will be influenced by the total diversity or number of different possible identifier sequences that are needed for a particular purpose.

In some cases, sequence units or nucleotide blocks may be used consecutively to form a single longer nucleotide block corresponding to the full identifier sequence, i.e. a series of consecutive sequence units or nucleotide blocks. The term “consecutive” is used to refer to sequential sequence units or nucleotides blocks in a polynucleotide which immediately follow the previous sequence unit or nucleotide block without intervening nucleotides. For example, an identifier sequence comprising 6 nucleotide blocks, wherein the nucleotide blocks are selected from di-adenosine, di-guanosine, di-cytidine, di-thymidine and di-uridine may have the sequence “AAGGCCTTAAGG”.

In other cases, one or more spacers or other sequence elements may be included between the sequence units or nucleotide blocks that make up the identifier sequence. The position or identity of the sequence units or nucleotide blocks of the identifier sequence in the polynucleotide may be determined by any suitable means, for example by adding the sequence units or nucleotide blocks at pre-determined positions in the polynucleotide or using nucleotide analogues in or otherwise marking or tagging the sequence units or nucleotide blocks of the identifier sequence. The identifier sequence or the region of the polynucleotide containing all of the sequence units or nucleotide blocks of the identifier sequence, optionally with other intervening sequence elements, may in some cases be up to 20, 22, 24, 26, 28, 30, 25, 40, 45, 50, 70, 100 or 200 nucleotides in length. A typical UMI built up from a series of 8 to 12 consecutive nucleotide blocks would be 16 to 25 nucleotides in length if blocks that are two nucleotides in length are used, or 24 to 36 nucleotides in length if blocks that are two nucleotides in length are used.

In some cases, one or more or each of the nucleotide block in a pool consists of two or more of the same nucleotide. For example, the nucleotide block pool may in some cases comprise or consist of the blocks AA (di-adenosine), TT (di-thymidine), GG (di-guanosine) and CC (di-cytidine) (or UU (di-uridine)) or/or the blocks AAA (tri-adenosine, TTT (tri-thymidine), GGG (tri-guanosine) and CCC (tri-cytidine) (or UUU (tri-uridine)); or the blocks AAA, TTT, CCX and GGY, wherein X is A, or otherwise T or G, and Y is C, or otherwise A or T; or any combination thereof. In other cases, one or more or each nucleotide block sequence may comprise a duplicate or triplicate or other multiple of a nucleotide analogue.

The term “nucleotide” as used herein, particularly in relation to the nucleotide blocks of an identifier sequence, may refer to natural or canonical nucleotides (i.e., “naturally occurring” or “natural” nucleotides), which include adenosine, guanosine, cytidine, thymidine and uridine, or to a non-canonical nucleotide or a nucleotide analogue. The polynucleotides or nucleotide blocks described herein may comprise any combination of natural nucleotides. Alternatively, any non-canonical nucleotides or nucleotide analogues may appear in one or more identifier sequence.

Generally, a nucleotide analogue contains a nucleic acid analogue, a sugar and a phosphate group, or variants thereof, and integrates into a polynucleotide chain in place of a natural nucleotide. Using nucleotide analogues in the identifier sequence may be particularly useful where the nucleotide analogues produce a more distinct signal when sequencing. The person skilled in the art is able to select appropriate nucleotide analogues or combinations of nucleotides/analogues to use in the identifier sequence. Examples of nucleotide analogues that may be included in the polynucleotides or nucleotide blocks described herein are as follows.

Peptide nucleotides, in which the phosphate linkage found in DNA and RNA is replaced by a peptide-like N-(2-aminoethyl)glycine. Peptide nucleotides undergo normal Watson-Crick base pairing and hybridize to complementary DNA/RNA with higher affinity and specificity and lower salt-dependency than normal DNA/RNA oligonucleotides and may have increased stability. Locked nucleotides (LNA), which comprises a 2′-O-4′-C-methylene bridge and are conformationally restricted. LNA form stable hybrid duplexes with DNA and RNA with increased stability and higher hybrid duplex melting temperatures. Propynyl dU (also known as pdU-CE Phosphoramidite, or 5′-Dimethoxytrityl-5-(1-Propynyl)-2′-deoxyUridine,3′-[(2-cyanoethyl)-(N,N-diisopropyl)]-phosphoramidite). An unlocked nucleotidec (UNA), which is an analogue of a ribonucleotide in which the C2′-C3′ bond has been cleaved. UNA form hybrid duplexes with DNA and RNA, but with decreased stability and lower hybrid duplex melting temperatures. LNA and UNA may therefore be used to finely adjust the thermodynamic properties of the polynucleotides in which they are incorporated. Triazole-linked DNA oligonucleotides, in which one or more of the natural phosphate backbone linkages are replaced with triazole linkages, particularly when click chemistry is used for synthesising the polynucleotide. A 2′-O-methoxy-ethyl base (2′-MOE), such as 2-Methoxyethoxy A, 2-Methoxyethoxy MeC, 2-Methoxyethoxy G and/or 2-Methoxyethoxy T. A 2′-O-Methyl RNA base. A 2′-fluoro base, such as fluoro C, fluoro U, fluoro A, and/or fluoro G. Other specific examples of nucleotide analogues that may be used include 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, 5-Methyl dC, 5-Nitroindole, 5-hydroxybutynl-2′-deoxyuridine (Super T) and 8-aza-7-deazaguanosine (Super G). Super T 2,6-Diaminopurine (2-Amino-dA) and/or 5-Methyl dC. A biotinylated nucleotide. In some cases a polynucleotide, nucleotide block or identifier sequence described herein may comprise at least two, or at least 3, 4, 5, 6, 7, 8, 9, 10, 12, 14, 16, 18, 20, 25, 30, 35, 40, or up to 2%, 5%, 10%, 15%, 20%, 25%, 30%, 35%, 40% or more nucleotide analogues and/or biotinylated nucleotides, or any one type of nucleotide analogue as described herein.

### Linkers

In some cases, the analyte capture polynucleotides and/or hairpin polynucleotides comprise a sequence element that is proximal to the micro-bead and cleavable to release the polynucleotide from the micro-particle. In some cases, the sequence is a linker that is light (e.g. UV light)-sensitive (photocleavage), that is temperature-sensitive or thermolabile (thermocleavage), or that is cleaved on chemical exposure (chemical cleavage) or enzymatically (enzymatic cleavage).

In some cases, the linker is cleaved enzymatically. The enzyme may be a site-specific endonuclease, such as a restriction endonuclease. Restriction endonucleases typically cleave one or both strands of a double-stranded polynucleotide, such as dsDNA. The linker may therefore comprise a sequence recognisable by a site-specific endonuclease, such as a restriction endonuclease. In some cases, the array comprises a sequence element, such as a short polynucleotide, that is complementary to and anneal to the sequence recognisable by a site-specific endonuclease on the polynucleotides in the array.

In some cases, the hairpin sequence does not comprise a sequence recognisable by a site-specific endonuclease, such as a restriction endonuclease. Hairpin sequences comprise double-stranded regions which may be recognised by restriction endonuclease and thus cleaved is a restriction endonuclease is present in the reaction mixture (for example that is present to cleave a linker between the micro-bead and the PCR handle of a polynucleotide in the array). In some cases, the hairpin sequence does not comprise a sequence recognisable by a site-specific endonuclease, such as a restriction endonuclease, wherein the sequence recognisable by a site-specific endonuclease is present elsewhere in the polynucleotide. In some cases, the hairpin sequence does not comprise a sequence recognisable by a site-specific endonuclease, such as a restriction endonuclease, wherein the restriction endonuclease is present in the reaction mixture. In some cases, the methods described herein do not comprise a site-specific endonuclease, such as a restriction endonuclease. In some cases, the methods described herein do not comprise a site-specific endonuclease, such as a restriction endonuclease, that recognises a sequence in the hairpin sequence of the polynucleotides.

### PCR Handle Sequences

A PCR handle sequence hybridizes to PCR oligonucleotide primers during a PCR reaction. Typically, a PCR handle sequence may be at least 15, 16, 17, 18, 19 or 20 nucleotides in length and/or up to and 21, 22, 23, 24, 25, 30 or 35 nucleotides in length, for example about 15 to 30, or 18 to 25 nucleotides. In some cases, the PCR handle sequence(s) may comprise one or more nucleotide analogues, such as analogues described herein, that form double-stranded hybrids with higher stability than natural nucleotides. In this case, the PCR handle sequence(s) could be shorter, such as at least 3, 4, 5, 6, 8, 9, 10, 11, 12, 13 or 14 nucleotides in length, provided that the PCR handle sequence(s) was capable of hybridizing to PCR oligo as described herein. Hence, a PCR handle sequence(s) may include nucleotide analogues as described herein.

In some cases, one or more of the PCR handle sequences may comprise or consist of one of the following sequences: 5′-GTGGTATCAACGCAGAGTAC-3′; 5′-GTCCGAGCGTAGGTTATCCG-3′. In some cases, one or more of the PCR handle sequences may comprise or consist of one of the following sequences: 5′-GTGGTATCAACGCAGAGTAC-3′; 5′-GTCCGAGCGTAGGTTATCCG-3′, 5′-AAGCAGTGGTATCAACGCAGAGTAC-3′ or 5′-TACACGACGCTCTTCCGATCT-3′. The PCR handles may be compatible with standard Illumina sequencing to eliminate the need for custom read 1 sequencing primers to read BC/UMI.

### Kits

In some cases, the invention relates to a kit. The kit may be for generating one or more libraries from one or more groups of analytes or from polynucleotides of mixed sequence from a sample. The kit may comprise a plurality of micro-beads as described herein. The invention also relates to the use of a kit of the invention as described herein for generating one or more libraries from one or more groups of analytes or from polynucleotides of mixed sequence from a sample. The kit may also comprise buffers, enzymes (for example a polymerase (such as those described herein) and/or a reverse transcriptase or template switch reverse transcriptase (such as Moloney Murine Leukemia Virus (MMLV) reverse transcriptase) and optionally suitable accompanying buffers) and other components used for generating a library as described herein.

In other cases, the kit may be for generating a plurality of micro-particles or polynucleotides as described herein. The kit may comprise (i) a plurality of polynucleotides, each comprising a (first) ligation linker (typically at the 5′ end) and an analyte capture region (typically at the 3′ end), as described herein; and (ii) a plurality of polynucleotides each comprising the (first) ligation linker (typically at the 5′ end) and a hairpin sequence (typically at the 3′ end), as described herein.

In some cases, the kit may comprise (i) a plurality of polynucleotides, each comprising a (first) ligation linker (typically at the 5′ end), a UMI sequence, and an analyte capture region (typically at the 3′ end), as described herein; and (ii) a plurality of polynucleotides each comprising the (first) ligation linker (typically at the 5′ end) and a hairpin sequence (typically at the 3′ end), as described herein. The presence of a UMI sequence (and a capture polynucleotide) of the polynucleotides comprising a hairpin sequence is not necessary. This results in more efficient production of nucleotides of the invention, as the bead-proximal polynucleotides as described herein may be shorter (due to the lack of a UMI) and thus are synthesised with greater efficiency and fidelity, and the polynucleotides comprising the hairpin sequence.

Polynucleotides (i) and (ii) may be mixed (for example in the ratios described herein) or in separate containers. The ligation linkers are suitable for joining the polynucleotides of the kit to a plurality of other polynucleotides comprising, typically in a 5′ to 3′ direction, (a) a PCR handle sequence, (b) a barcode sequence, (c) a (typically 3′ terminal) second ligation linker; and optionally other sequence features as described herein, wherein the first and second ligation linkers are suitable for ligating together.

For example, the ligation linker(s) may be click-reactive groups, as are known in the art, for example an azide (or other 1,3-dipole) and an alkyne. Squaramide, urea, amide, CuAAC, phosphoramidate, phosphorothioate and thiol-thiol coupling reactions can also be used to generate artificial backbones that are recognised and read-through by DNA polymerases. Suitable linkers are described in Shivalingam et al. (2020), Angew. Chem. Int. Ed.59:28.

The ligation linker(s) may comprise a polynucleotide sequence. The ligation linker(s) described herein may be complementary or substantially complementary to a portion of a splint oligonucleotide. For example, where the ligation linker is on the 5′ end of a polynucleotide, the ligation linker comprises a sequence that is complementary or substantially complementary to a 3′ portion of the splint oligonucleotide. Where the ligation linker is on the 3′ end of a polynucleotide, the ligation linker typically comprises a sequence that is complementary or substantially complementary to a 5′ portion of the splint oligonucleotide. Where the ligation linker(s) comprise a polynucleotide sequence, the ligation linker(s) may be suitable for enzymatic ligation. The enzymatic ligation may be splint ligation. The enzymatic ligation may be catalysed by a DNA ligase, such as a T4 DNA ligase. The kit may therefore further comprise a splint oligonucleotide. The splint oligonucleotide typically comprises portions that are complementary to the first and second ligation linkers, e.g. such that the splint oligonucleotide is capable of hybridising to the polynucleotide comprising the first ligation linker and the polynucleotide comprising the second ligation linker, as shown in FIG. 7. The kit may further comprise a DNA ligase enzyme, such as T4 DNA ligase. The kit may further comprise one or more exonuclease enzymes to degrade splint oligonucleotide present in dsDNA form.

Advantages of splint ligation include the use of the ligation linkers as ‘spacers’ to help identify barcode and/or UMI sequences as described herein. Advantages associated with the use of click chemistry include the omission of ‘extra’ bases at the ligation site.

In some cases, the ligation linkers do not comprise a sequence recognisable by a site-specific endonuclease, such as a restriction endonuclease. In some cases, the ligation linkers do not comprise a sequence recognisable by a site-specific endonuclease, such as a restriction endonuclease, wherein the sequence recognisable by a site-specific endonuclease is present elsewhere in the polynucleotide. In some cases, the ligation linkers do not comprise a sequence recognisable by a site-specific endonuclease, such as a restriction endonuclease, wherein the restriction endonuclease is present in the reaction mixture. In some cases, the methods described herein do not comprise a site-specific endonuclease, such as a restriction endonuclease, that recognises a sequence in the ligation linkers.

The kit may further comprise a plurality of micro-particles, wherein each micro-particle comprises a micro-bead and an array of polynucleotides, wherein each polynucleotide comprises, in a 5′ to 3′ direction: (a) a PCR handle sequence; (b) a barcode sequence, wherein the barcode sequence of each polynucleotide of a micro-particle is the same as the barcode sequence of each other polynucleotide of the same micro-particle; and wherein the array of polynucleotides of each micro-particle has a different barcode sequence from the array of polynucleotides of essentially each other micro-particle; and (c) a (typically 3′ terminal) second ligation linker (suitable) for ligation to the first ligation linker. The kit may further comprise suitable buffers and/or enzymes or other components that are useful for ligating polypeptides comprising the first ligation linker to other polypeptides, e.g. those comprising the second ligation linker.

In some embodiments, the array of polynucleotides on the micro-bead do not comprise a UMI. In such cases, the UMI may be present on the polynucleotides of the kit comprising the analyte capture region.

The kits may also include instructions for use of the kit, for example instructions for carrying out any appropriate method described herein using the kit.

### Micro-Particle Synthesis

The invention provides methods for producing a micro-particle or a plurality of micro-particles as described herein.

The method comprises providing (i) a plurality of polynucleotides each comprising a first ligation linker and an analyte capture region; and (ii) a plurality of polynucleotides each comprising the first ligation linker and a hairpin sequence. The polynucleotides may have any of the features described herein. The method further comprises providing a micro-particle, wherein the micro-particle comprises a micro-bead and an array of polynucleotides, wherein each polynucleotide of the array comprises, in a 5′ to 3′ direction: (a) a PCR handle sequence; (b) a barcode sequence, wherein the barcode sequence of each polynucleotide in the array is the same as the barcode sequence of each other polynucleotide in the array; and (c) a second ligation linker for ligation to the first ligation linker. The micro-particle may have any other suitable features described herein. The first and second ligation linkers are suitable for ligating together. For example, the ligation linker(s) may be click-reactive groups, as are known in the art, for example an azide (or other 1,3-dipole) and an alkyne, or as described in Shivalingam et al. (2020). The method further comprises contacting the micro-particle with a mixture of the plurality of polynucleotides (i) and the plurality of polynucleotides (ii) under conditions suitable for ligating the first ligation linker to the second ligation linker. Where the method is applied to produce a plurality of micro-particles, then typically the barcode sequence of each polynucleotide in an array is the same as the barcode sequence of essentially each other polynucleotide in the array of a micro-particle; and wherein the array of polynucleotides of each micro-particle has a different barcode sequence from the array of polynucleotides of essentially each other micro-particle. Where the ligation linker(s) are nucleotide sequences as described herein, such as those comprising complementarity to a splint oligonucleotide, the method may comprise contacting the micro-particle with a mixture of the plurality of polynucleotides (i), the plurality of polynucleotides (ii), and a splint oligonucleotide, under conditions for hybridising the splint oligonucleotide to the first and second ligation linkers. The method may further comprise contacting the hybridised polynucleotides with a DNA ligase. The method may further comprise removing splint oligonucleotide by heat-washing, exonuclease treatment and/or other clean-up methods known in the art.

The invention also provides micro-particles or a plurality of micro-particles produced by the method. Typically, the barcode sequence of each polynucleotide in an array is the same as the barcode sequence of essentially each other polynucleotide in the array of a micro-particle; and the array of polynucleotides of each micro-particle has a different barcode sequence from the array of polynucleotides of essentially each other micro-particle. However, it is sufficient that each barcode sequence of the polynucleotide array of each micro-particle is different to each barcode sequence of the polynucleotide array of each other micro-particle.

Typically, the plurality of polynucleotides each comprising a first ligation linker and an analyte capture region and the plurality of polynucleotides each comprising the first ligation linker and a hairpin sequence are mixed together before contact with the micro-particle(s).

In some cases the ratio polynucleotides comprising a first ligation linker and an analyte capture region to polynucleotides comprising the first ligation linker and a hairpin sequence is 1000000:1 to 1:100, or 100000:1 to 1:100, or 10000:1 to 1:100, or 100:1 to 1:100, or 10:1 to 1:100; or 1000000:1 to 1:10, or 100000:1 to 1:10, or 10000:1 to 1:10, or 1000:1 to 1:10, or 100:1 to 1:10; 1000000:1 to 1:1, or 100000:1 to 1:1, or 10000:1 to 1:1, or 100:1 to 1:1, or 10:1 to 1:1.

### Analyte Capture and Library Generation

In some embodiments, the invention provides a method of generating a library of analytes from a plurality of samples. Any suitable samples may be used. Examples include single cells (for example bacterial cells, animal cells, plant cells, fungal cells, mammalian cells or human cells), single cell nuclei, cell vesicles (for example exosomes or mini-vesicles) or other compartments enclosed by a lipid membrane. Biological samples may for example be obtained from a biological tissue or fluid, such as blood, a blood fraction, serum, plasma, saliva or urine. In some cases, the method may comprise isolating the single cell, nucleus, vesicle or other compartment, or a lysate thereof and/or preparing a suspension of single cells/nuclei/vesicles or other components making up each single sample. In other cases, the plurality of samples may relate to a plurality of discrete spatial positions within a two or three dimensional sample, such as a tissue sample/section.

The method of library generation relies on contact being made between the analytes of individual samples and individual micro-particles as described herein, or the polynucleotides thereof, on which the analytes may be captured. Hence, the method comprises a step of dividing both the samples (or the analytes thereof) and the micro-particles (or the sets of polynucleotides thereof), between the same set of (plurality of) compartments. The compartments are typically fluidic compartments. For example, each compartment could be a well (or a fluidic compartment contained in a (solid) well), such as a well in a multi-well plate, microplate or slide, a discrete site/position on a microfluidic chip, or a (micro-) droplet/aqueous (micro-) droplet (i.e. the internal phase), for example in an emulsion with oil (i.e. the external phase). Such compartments/droplets may be conveniently generated using a microfluidics device, as are known in the art.

Both sample/analytes and micro-particle/polynucleotides thereof are added to a compartment, simultaneously or in either order. In a typical example, for example where a microfluidics device is used, both the samples (e.g. single cells) and the micro-particles are provided in an aqueous solution. The two solutions may be mixed together and the mixed solution divided into compartments. Alternatively, each solution can be divided into a plurality of compartments, e.g. droplets, for example in a microfluidics device, and single compartments/droplets of each solution are joined together to form new compartments/droplets each of which may contain sample, micro-particle/polynucleotides thereof, or both. The micro-particle/polynucleotides thereof are typically “super-loaded” into the compartments in that at least some compartments may contain more than one micro-particle/set of polynucleotides thereof. Other compartments may contain no micro-particle/set of polynucleotides thereof. However, it will generally be desirable to limit the number of compartments that contain sample/analytes but no micro-particle/polynucleotides thereof to reduce sample loss from an experiment/analysis. For example, typically at least 40%, or at least 50%, 60%, 70%, 80%, 90%, 95%, 98% or 99% of sample, or between about 40% or 50%, 60%, 70%, 80%, 90% or 95% and 100% of sample, is contacted with at least one micro-particle/polynucleotides thereof in a compartment. Most typical is about 50% or 60% to 100%. Typically, the average number of micro-particles/sets of polynucleotides thereof per compartment may be between 0.4 and 10, or between 0.5, 1, 1.5, 2, 3, 4 or 5 and 5, 6, 7, 8, 9 or 10 micro-particles/sets of polynucleotides thereof per compartment. Most typically, the average number of micro-particles/sets of polynucleotides thereof per compartment is between 1 and 4. The skilled person is able to calibrate the number of compartments, the number of micro-particles and number of samples, such that micro-particles/sets of polynucleotides thereof and sample come into contact within at least a proportion of compartments at the desired frequency. In some cases, there may be at least 500, or at least 5,000, 25,000, 50,000, 100,000, 200,000, for example between 500 and 3 million, or between 2,000 and 1.5 million, or, most typically, between 50,000 and 400,000 separate compartments.

Sample/sample analytes and micro-particles/polynucleotides thereof come into contact within individual compartments containing both. In some cases where the samples include a lipid membrane, the membrane may be broken (e.g. cells lysed) before or after contact with the micro-particle or array of polynucleotides. In some cases, the polynucleotides of a micro-particle may be cleaved from the surface of the micro-particle or a micro-particle hydrogel may be dissolved to release the polypeptides from (the bead of) a micro-particle before or after contact with the sample/analyte, for example by exposing the micro-particles to UV light or heat, or contacting the micro-particle with appropriate chemicals or enzyme(s), for example as described elsewhere herein. In some cases, it may be convenient to include the micro-particles/polynucleotides thereof in a solution comprising a suitable cell/membrane lysis buffer, and/or to include the samples/analytes in a solution/buffer comprising agents for cleavage of the linker/dissolving of the hydrogel. For example, a microfluidics device may be used to join two aqueous flows into discrete microfluidic droplets. One flow may comprise a single cell or single cell nuclei suspension in cell buffer and optionally chemicals or enzymes needed to cleave the polynucleotide linker. The other flow may comprise a suspension of micro-particles as described herein, optionally in cell/membrane lysis buffer. Some of the droplets that are formed comprise both a sample/analytes and micro-particle/polynucleotides thereof, resulting in contact between the analytes and the array of polynucleotides.

Other components needed for downstream reactions and processes may be included in the sample/analyte solution and/or the micro-particle/polynucleotide thereof solution. For example, the sample/analyte solution may comprise template switch oligonucleotides and/or the micro-particle/polynucleotide thereof solution may comprise reverse transcriptase, in particular when the 3′ analyte capture region of the polynucleotides is an RNA capture region, such as a polythymidine.

The compartments are incubated under conditions suitable for binding between analytes and the analyte capture regions of the analyte capture polynucleotides, and for the hairpin sequences of different polynucleotides to dimerise. Typically, the same conditions can be used for both. For example, the compartments may be heated to melt/denature the hairpin structures. Such heating could also be useful for removing secondary structure in analytes such as RNA analytes and hence improving subsequent capture rates. A heating step could also cause or increase cell lysis or membrane disruption where necessary to bring the micro-particles/polynucleotides thereof into contact with analytes enclosed within a membrane/cell membrane/nuclear membrane and the like. A heating step could also cause or increase release of polynucleotides from the micro-particle/bead, e.g. by thermal cleavage or dissolving of a hydrogel, for example as described herein. This heating step is typically to about 70° C., or between about 30° C., 40° C., 5° C. or 60° C. and about 70° C. Most typically the heating step is to between about 40° C. and about 70° C. However, the temperature needed to melt/denature the hairpin structure will depend on the design of the hairpin sequence itself, for example longer hairpin sequences generally melt/denature at higher temperatures. Those in the art are able to design suitable hairpin sequences and choose a suitable melting temperature. The temperature is generally kept below around 70° C. (for example below 71° C.) to avoid denaturing a reverse transcriptase that is typically included in the compartment for a downstream reverse transcription step. However, a higher temperature could be used if otherwise tolerated by the method overall, for example if the reverse transcriptase is not included at this stage and is only added later. The heating step may typically last for about 1 minute to 1 hour, about 2 to 30 minutes, or more typically about 4 to 10 minutes (or about 5 minutes). The compartments are then cooled, typically to about room temperature (about 18 to 23° C.). As the compartments cool, the hairpin sequences of different polynucleotides will preferentially dimerise (i.e. across their full complementary/palindromic region) rather than reform hairpins because the dimer comprises more base pairs. In some cases, repeated cycles of heating (typically above the melting temperature of the hairpin(s) but below the melting temperatures of the dimers) and cooling could be used to maximise the desired binding. At the same time, analytes will in many cases bind to the analyte capture regions, i.e. under the same conditions used to dimerise hairpin sequences. However, in some embodiments where optimal or necessary conditions for dimerising hairpins and binding analytes are different, then the compartments may be subjected to different conditions sequentially and in either order. Capture of analytes by the analyte capture polypeptides may otherwise be achieved using conditions and methods known in the art and/or described herein.

The polynucleotides may be released from the micro-particles/beads as described herein at any suitable stage. This is typically before or during hairpin dimerization and/or analyte capture because polynucleotides that are free in solution and not bound to a micro-bead are expected to dimerise and/or capture analyte more efficiently. Library preparation of captured analytes may subsequently proceed according to known or conventional methods. For example, RNA analyte may be bound by a polythymidine analyte capture region, reverse transcribed using the bound RNA as template to provide an RNA/cDNA hybrid (most typically whilst still in compartments/before compartments are combined), then template switch oligonucleotides may be added at the end of the RNA/cDNA hybrid and used together with the PCR handle sequence to amplify the analyte sequence together with the BC tag. After amplification, the PCR products may be sequenced to identify the analyte and determine the BC tag. The separate compartments may be combined at any suitable stage to simplify downstream processing, as is well known in the art, for example by breaking an emulsion.

Likewise, the dimers and the capture polynucleotides/analyte-bound polynucleotides may be separated at any suitable stage for more efficient downstream processing. For example, separating dimers and the capture polynucleotides/analyte-bound polynucleotides may be performed after combining the compartments/breaking an emulsion (for example after in-compartment/droplet reverse transcription, and before PCR amplification). Separation could occur either before or after nuclease treatment to remove un-dimerised hairpins and unbound capture polynucleotides. Separation could, for example, be achieved by using hairpin sequences with incorporated biotin (made, for example, using biotin dT during synthesis) and then streptavidin-coated magnetic beads, provided that the analyte capture polynucleotides do not also include biotin, as described herein. Biotin would separate all of the hairpin (if not already digested) and dimer sequences from the analyte capture polynucleotides (if not already digested) and analytes bound to capture polynucleotides. Another option could be separation based on size, using, for example, AMPure XP beads or the like. Size separation would effectively separate capture polynucleotides bound to analyte/cDNA derived therefrom from hairpin dimers, but not hairpin dimers and un-bound analyte capture polynucleotides where these are a similar size.

Processing of the dimers typically involves filling in the single-stranded overhangs of the dimerised polynucleotides to produce a set of double stranded polynucleotides comprising the hairpin sequence flanked by two barcode sequences. This can be done, for example, using reverse transcription using the free 5′ ends of the dimers as template. Hence, in some cases reverse transcription of analyte and dimers may be achieved in a single step. The reverse transcription step may typically be carried out in the individual compartments, e.g. in droplet reverse transcription. Typically, the different compartments are combined after the reverse transcription/5′ overhang fill step for combined/in bulk further downstream processing. However, in other cases the compartments may be combined prior to the reverse transcription/5′ overhang fill step.

In some cases, polynucleotides comprising the hairpin sequence/hairpin polynucleotides that have not dimerised are removed from the compartments or the combined mixture, for example using a single-strand specific endo- or exonuclease. This is advantageous because undimerised hairpins could randomly combine, e.g. after compartments are combined, during subsequent PCR amplification, or act as PCR primers for oligos that had dimerised. The exonuclease may be a single-strand specific 5′ exonuclease, for example RecJf.

The polynucleotides comprising a hairpin sequence and two barcode sequences may be amplified (using the PCR handle sequences/PCR) and sequenced using conventional means known in the art.

### Bead-Hashing and Digital Analysis

After sequencing, analytes that are associated with the same barcode can be identified digitally. Using prior art methods, groups of library analytes that came from a single sample, but where the sample was co-compartmentalised during library preparation with more than one analyte capture micro-particle, can be wrongly attributed to multiple separate samples based on the multiple different barcode sequences associated with the different co-compartmentalised micro-particles. According to the invention, the barcode sequences in the hairpin polynucleotides associated with each micro-particle can be used to signpost groups of analytes that were captured in the same compartment, even if they were captured by polynucleotides from different micro-particles.

The method involves sequencing the polynucleotides having the hairpin sequence and two barcode sequences. If only one micro-particle/set of polypeptides associated therewith was compartmentalised with a sample, then all of the analytes from the sample will have the same barcode sequence. Moreover, all of the sequenced hairpin polynucleotides that share the same barcode sequence will have two copies of the same barcode sequence. Hence when a barcode sequence is only found in duplicated pairs in the sequenced hairpin polynucleotides, and never in a single copy and in combination with a different barcode sequence, then all of the analytes having the same barcode sequence can be assumed to have come from sample that was co-compartmentalised during library preparation with a single micro-particle/set of polypeptides thereof.

Hence, the method of the invention may comprise identifying a captured analyte from a sample that was co-compartmentalized during library generation with one micro-particle only, or the arrays of polynucleotides thereof, the method comprising: (I) identifying a polynucleotide comprises the hairpin sequence and two copies of the same barcode sequences/two identical barcode sequences that was produced during library preparation as described herein; (II) identifying all, or substantially or essentially all of the polynucleotide that comprise the hairpin sequence and barcode sequence of the polynucleotide identified in step (I); (III) determining that all of the polynucleotides identified in step (II) comprise two copies of the same barcode sequence; (IV) identifying an analyte captured by a polypeptide that shares the same barcode sequence as the polynucleotides identified in steps (I) and (II); and (V) identifying the analyte identified in step (IV) as being from a sample that was co-compartmentalized during library generation with one micro-particle only, or the arrays of polynucleotides thereof. The method may further comprise identifying all such analytes in the library or all such analytes sharing the same barcode.

When two micro-particles/sets of polypeptides associated therewith are co-compartmentalised during library preparation, then hairpin dimers having three different combination of barcode sequences will be generated: 1) hairpin dimers having two copies of the barcode sequence associated with one of the micro-particles/sets of polynucleotide thereof; 2) hairpin dimers having two copies of the barcode sequence associated with the other one of the two micro-particles/sets of polynucleotide thereof; and 3) hairpin dimers having one barcode sequence associated with one of the two micro-particle s/polynucleotides thereof and one barcode sequence associated with the other of the two micro-particle s/polynucleotides thereof, i.e. having two different barcode sequences. However, all of the hairpin dimers having either one of these barcodes will only appear in one of these three combinations, and not in combination with any other barcode sequence.

Hence, the method of the invention may comprise identifying a captured analyte from a sample that was co-compartmentalized during library generation with two different micro-particles only, or the arrays of polynucleotides thereof, the method comprising: (I) identifying a polynucleotide that was produced during library preparation as described herein, and that comprises the hairpin sequence and two different barcode sequences; (II) identifying all, or substantially or essentially all of the polynucleotide that comprise the hairpin sequence and either of the two barcode sequences of the polynucleotide identified in step (I); (III) determining that all of the polynucleotides identified in step (II) do not comprise any barcode sequence other than the two barcode sequences of the polynucleotide identified in step (I); (IV) identifying an analyte captured by a polypeptide that shares the either one of the two barcode sequences as the polynucleotides identified in steps (I) and (II); and (V) identifying the analyte identified in step (IV) as being from a sample that was co-compartmentalized during library generation with two micro-particles only, or the arrays of polynucleotides thereof. The method may further comprise identifying all such analytes in the library or all such analytes sharing the same one or two barcodes.

If steps (II) and (III) are not carried out, then it will not immediately be known whether a library analyte that has either of the two barcode sequences of the polynucleotide identified in step (I) (or the sample from which it was derived) was co-compartmentalised with two micro-particles/sets of polynucleotides thereof only, or with more than two micro-particles/sets of polynucleotides thereof. However, any such analyte was co-compartmentalised with multiple micro-particles/sets of polynucleotides thereof.

Hence, the method of the invention may comprise identifying a captured analyte from a sample that was co-compartmentalized during library generation with multiple micro-particles, or the arrays of polynucleotides thereof, the method comprising: (I) identifying a polynucleotide that was produced during library preparation as described herein, and that comprises the hairpin sequence and two different barcode sequences; (II) identifying an analyte that was captured by a polynucleotide that shares either of the two barcode sequences with the polynucleotide identified in step (I); and (III) identifying the captured analyte identified in step (II) as being from a sample that was co-compartmentalized during library generation with multiple different micro-particles, or the arrays of polynucleotides thereof.

The invention also provides a method of identifying a set of analytes (or, in some cases, all of the analytes), or the sample from which they were derived, that were co-compartmentalised during library generation, e.g. with multiple micro-particles. The method comprises (I) identifying a set of polynucleotides that were produced during library preparation as described herein and that each comprise the hairpin sequence and two different barcode sequences, wherein all of said barcode sequences in the set of polynucleotides share a string of different combinations of the barcode sequences between the polynucleotides of the set; (II) identifying a set of analytes captured by polynucleotides that share any of the barcode sequences with the set of polynucleotides identified in step (I); and (III) identifying the set of analytes identified in step (II) as being from sample that was co-compartmentalized during library generation with multiple micro-particles, or the arrays of polynucleotides thereof.

The invention also provides a method of determining how many micro-particles/sets of polynucleotides thereof that a library analyte (or the sample from which it was derived) was co-compartmentalised with. The method comprises: (I) determining the barcode sequence associated with the analyte (or the capture polynucleotide that captured the analyte); (II) identifying all, or substantially or essentially all of the polynucleotides produced during library preparation as described herein and that comprise the hairpin sequence and the same barcode sequence as the analyte; (III) determining the number of different barcode sequences comprised in the set of hairpin polynucleotides identified in step (II); and (IV) determining that the number determined in step (III) is the micro-particles/sets of polynucleotides thereof that the library analyte (or the sample from which it was derived) was co-compartmentalised with during library generation.

### EXAMPLES

### Example 1—Dimer Formation by Hairpin Sequences

The following oligos were designed:

Oligo (BH-BC2):
AAGCAGTGGTATCAACGCAGAGTACGCCCGGGCCAGANNNNNNNNgac
ttaagaattcttaagtc
SMART
BARCODE
UMI
hairpin

A solution of BH-BC2 oligos (at 10 μM) was heated to 70° C. for 5 minutes, then cooled to room temperature.

The following 6 reactions were set up using the annealed oligo:
1. No enzyme control: 1 μl Oligo, 49 μl H2O
2. T4 DNA polymerase: 5 μl buffer 2.1, 2.5 μl 100 μM dNTPs, 1 μl Oligo, 1 μl T4 DNA pol, 40.5 μl H2O; incubate 15 minutes @ 12 C, then add 1 μl 0.5M EDTA and incubate @75 C for 20 minutes
3. T4 DNA polymerase: 5 μl buffer 2.1, 2.5 μl 100 μM dNTPs, 1 μl Oligo, 1 μl T4 DNA pol, 40.5 μl H2O; incubate 30 minutes @ 12 C, then add 1 μl 0.5M EDTA and incubate @75 C for 20 minutes
4. Kappa Hifi: 25 μl Kappa Hifi master mix, 1 μl Oligo, 24 μl H2O; incubate 5 minutes @ 72 C
5. RT (reverse transcriptase): 10 μl 5× RT buffer, 10 μl 20% ficoll, 5 μl 100 uM dNTPs, 1.25 μl Nxgen RNAse inhibitor, 2.5 μl Maxima H-RT, 1 μl Oligo, 20.25 μl PBS; incubate 30 minutes @ room temperature then 90 minutes @ 42 C
6. Phi29 DNA polymerase: 5 μl 10× buffer, 5 μl 100 μM dNTPs, 1 μl BSA, 1 μl phi29, 1 μl Oligo, 37 μl H2O; 10 minutes @ 30 C then 65 C for 10 minutes At completion of respective incubations, reactions were cleaned up with 90 μl (1.8×) SPRIselect beads and run on the tapestation (D1000 HS).

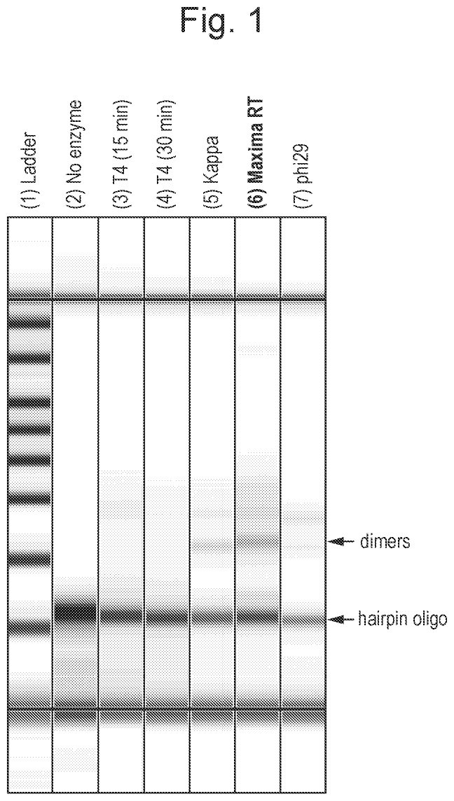

The results are shown in FIG. 1. Dimers of the expected size could be seen most strongly with RT, to a lesser extent with Kappa Hifi and faintly with phi29. In all reactions, the majority of the oligo remained in monomer (hairpin) form.

### Example 2—Nuclease Treatment Remove Hairpin Oligos Without Destroying Dimers/cDNA

The following 5 reactions were set up using the RT reaction from Example 1:
1. Hairpin control: same as 1 in Example 1.
2. RT only: same as 5 in Example 1.
3. RT+RecJf: RT reaction from 5 above in 20 μl after cleanup, 5 μl 10× buffer, 3 μl RecJf enzyme, 22 μl H2O; 37 C for 20 minutes then 65 C for 20 minutes
4. RT+MBN: RT reaction from 5 above in 20 μl after cleanup, 5 μl 10× buffer, 3 μl MBN enzyme, 22 μl H2O; 30 C for 30 minutes
5. RT+Exo I: RT reaction from 5 above in 20 μl after cleanup, 5 μl 10× buffer, 3 μl Exo I enzyme, 22 μl H2O; 37 C for 15 minutes then 80 C for 15 minutes

At completion of respective incubations, reactions were cleaned up with 90 ul (1.8×) SPRIselect beads and run on the tapestation (D1000 HS).

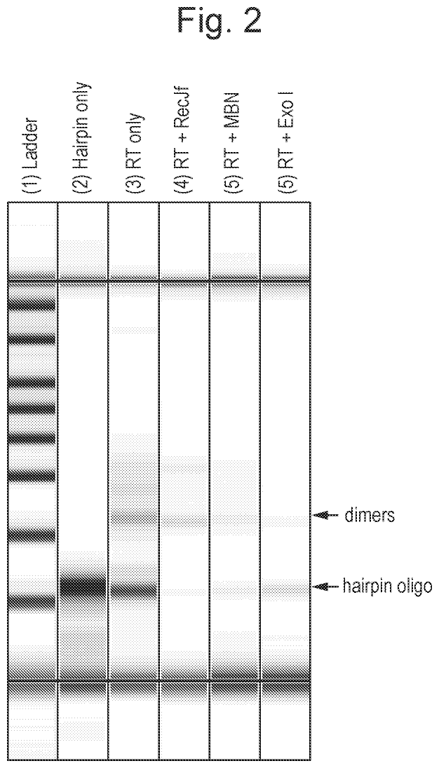

The results are shown in FIG. 2. Given that the melting temperature of the hairpin region is 26 C, the incubation temperatures of the various single-strand specific nucleases should ensure the entire length of the monomers are available for digestion, whereas dimer molecules would remain hybridised at these temperatures and remain protected. As with Example 1, dimers could be seen following RT, but much of the oligo remained as monomer hairpins. All three nucleases heavily reduced the amount of hairpin detected, although Exo I also removed some of the dimers. RecJf (a single-strand specific 5′ exonuclease) gave the greatest removal of hairpins, while retaining the most dimer band. MBN (mung bean nuclease; a single-strand specific DNA and RNA endonuclease) gave intermediate results. These results could be optimisation (e.g. by increased enzyme concentration and/or increase digestion time). Hence, use of these enzymes, especially RecJf is viable for the removal on undimerised hairpins, e.g. post emulsion breakage, but prior to PCR amplification.

### Example 3—Heterodimers can be Detected After PCR Amplification

Two different hairpin pairs were heated to 70 C for 5 minutes and cooled to room temperature:

Pair 1:
BH_oligoA_HP12:
AAGCAGTGGTATCAACGCAGAGTACagattgggacgcNNNNNNNNgacttataagtc
BH_oligoB_HP12:
AAGCAGTGGTATCAACGCAGAGTACgcaagaaaaaatcacgacttgtaagtggttacNNNNNNNNgact
tataagtc
Pair 2:
BH_oligoA_HP20:
AAGCAGTGGTATCAACGCAGAGTACagattgggacgcNNNNNNNNgacttaagaattcttaagtc
BH oligoB HP20:
AAGCAGTGGTATCAACGCAGAGTACgcaagaaaaaatcacgacttgtaagtggttacNNNNNNNNgact
taagaattcttaagtc

Key:
SMART
stuffer
UMI
hairpin

In pair 1, the hairpin region is 12 base pairs long, whereas the hairpin region is 20 base pairs long in pair 2. In both pairs oligo A has a 12 base pair stuffer region, whereas oligo B has a 32 bp stuffer region. Thus, within each pair, 3 different sized products are expected after RT and PCR: A:A homodimers will be the smallest, A:B heterodimers will be 20 bp bigger and B:B homodimers will be 20 bp larger again. Dimers should be formed in a 1:2:1 ratio. Reactions were setup as follows to mimic the RT conditions of a Drop-seq experiment: 10 μl 5× RT buffer, 10 μl 20% ficoll, 1.25 μl 10% IGEPAL, 5 μl 100 μM dNTPs, 1.25 μl Nxgen RNAse inhibitor, 2.5 μl Maxima H-RT, 19 μl H2O, 0.5 μl oligo A, 0.5 μl oligo B Reactions were heated to 70 C for 5 minutes, then 22 C for 30 minutes and 42 C for 90 minutes. Samples were cleaned up with 1.8× SPRIselect beads and eluted in 20 μl H2O, which was then combined with 5 μl 10× buffer, 3 μl RecJf enzyme, 22 μl H2O and incubated at 37 C for 20 minutes then 65 C for 20 minutes. Samples were cleaned up again with 1.8× SPRIselect beads and eluted in 20 μl H2O, which was then combined with 25 μl Kappa Hifi and 5 μl of 2 μM SMART primer (AAGCAGTGGTATCAACGCAGAGT) and amplified for 12 cycles. At completion of PCR, reactions were cleaned up with 0.9× SPRIselect beads and run on the tapestation (D1000 HS).

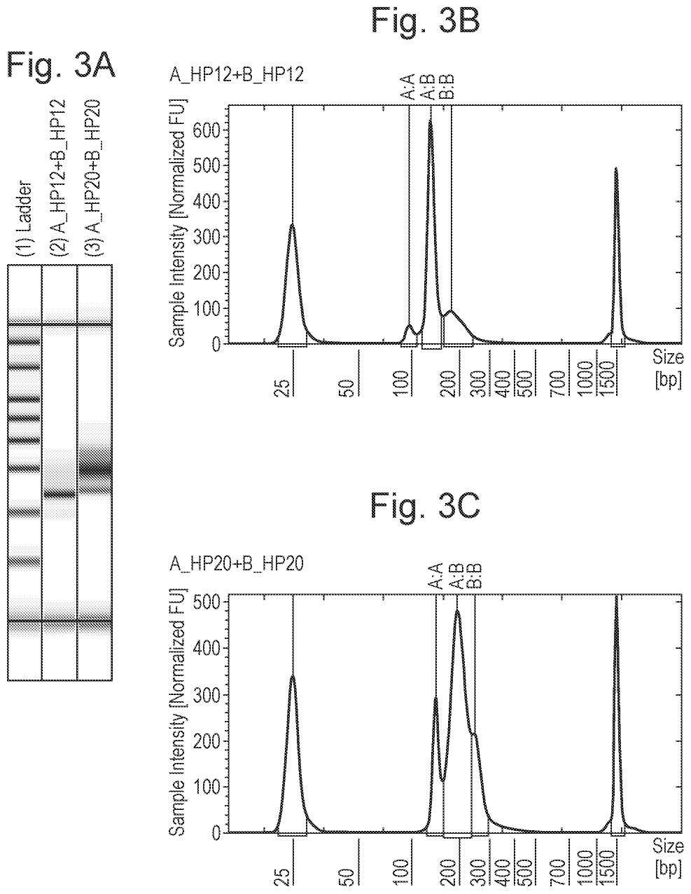

The results are shown in FIG. 3. For both oligo pairs, three bands are observed, with no contaminating monomer hairpin peak. Although sizes of the peaks were a little large than expected, this may reflect that the Tapestation was run with an electronic ladder rather than a true molecular weight marker. As expected, in both cases, the heterotrimer was the dominant peak.

### Example 4—Generation of Micro-Particles Using Enzymatic Ligation

The inventors devised a method of ligation of a free polynucleotide comprising a UMI sequence and an analyte capture region to a micro-particle associated polynucleotide comprising a PCR handle and a barcode sequence. This allows the final sequence to be synthesised as two shorter oligos, which would be expected to improve overall fidelity, plus the free oligo can be purified to remove truncated oligos, since truncation of the UMI can be a problem when oligos are added to beads in one continuous synthesis.

The bead sequence was:

Bead BC:
5′-Bead-HEG-Linker-PC-
TCTCTCTAAGCAGTGGTATCAACGCAGAGTACNNNNNNNNNNNNGGTGT
AATGA

Bold bases are the PCR handle. The Ns represent the barcode region. The underlined sequence provides one half of a splint region to allow ligation.

The sequence of the free polynucleotide comprising a UMI sequence and an analyte capture region was:

freeUMI:
5′-P-GGTCGTAGTGNNNNNNNNNNNNNNNNNNNNNNNNVTTTTTTTTT
TTTTTTTTTTTTTTTTTTT*T*T-3′

Underlined bases are the other half of the splint region. N's represent the UMI. * =phosphorothioate bonds to protect from exonuclease digest.

The splint oligonucleotide sequence was CACTACGACCTCATTACACC.

Splint ligation reactions were performed using T4 DNA ligase at room temperature for 4 hours. Splint oligonucleotide was removed using hot-wash (wash solution at 65° C.). PCR amplification was performed and representative gels are shown in FIG. 8A. The results indicate that ligation is occurring with good efficiency.

cDNA was generated using beads synthesised via splint ligation as described. The ligated beads were contacted with an RNA sample and reverse transcription reagents to produce cDNA. PCR was then performed to amplify the cDNA. The amount and approximate size of amplified cDNA following 10 cycles of PCR was determined and is shown in FIG. 8B.

Whilst the enzymatic ligation performed in Example 4 only involves the ligation of an analyte capture region, the methods can be easily adapted to the present invention. For example, multiple different 3′ ends (comprising analyte capture regions or hairpin sequences) can be mixed in their desired ratio to result in beads comprising polynucleotides having different 3′ ends in the desired ratio.

1. A micro-particle comprising a micro-bead and an array of polynucleotides, wherein each polynucleotide of the array comprises, in a 5′ to 3′ direction:
(a) a PCR handle sequence;
(b) a barcode sequence, wherein the barcode sequence of each polynucleotide in the array is the same as the barcode sequence of essentially each other polynucleotide in the array; and
(c) an analyte capture region on a proportion of the polynucleotides and a hairpin sequence on a proportion of the polynucleotides.

2. The micro-particle of claim 1, wherein the polynucleotides are conjugated to the surface of the micro-bead at the 5′ end of the polynucleotides.

3. The micro-particle of claim 1 or 2, wherein the hairpin is at the 3′ end of the polynucleotides.

4. The micro-particle of any one of claims 1 to 3, wherein the polynucleotides are conjugated to the surface of the micro-bead at the 5′ end of the polynucleotides and the hairpin is at the 3′ end of the polynucleotides

5. The micro-particle of any one of claims 2 to 4, wherein each polynucleotide of the array further comprises a cleavable-linker, optionally a photocleavable linker, between the 5′ end conjugated to the micro-bead and the PCR handle sequence.

6. The micro-particle of any one of claims 1 to 5, wherein each polynucleotide in the array comprises a single barcode sequence.

7. The micro-particle of any one of claims 1 to 6, wherein each polynucleotide in the array exclusively comprises either an analyte capture region or a hairpin sequence.

8. A plurality of micro-particles, wherein each micro-particle comprises a micro-bead and an array of polynucleotides, wherein each polynucleotide of the array comprises, in a 5′ to 3′ direction:
(a) a PCR handle sequence;
(b) a barcode sequence; and
(c) an analyte capture region on a proportion of the polynucleotides and a hairpin sequence on a proportion of the polynucleotides;
and wherein each barcode sequence of the polynucleotide array of each micro-particle is different to each barcode sequence of the polynucleotide array of each other micro-particle.

9. A kit comprising (i) a plurality of polynucleotides, each comprising a first ligation linker and an analyte capture region; and (ii) a plurality of polynucleotides each comprising the first ligation linker and a hairpin sequence.

10. The kit of claim 9, further comprising a plurality of micro-particles, wherein each micro-particle comprises a micro-bead and an array of polynucleotides, wherein each polynucleotide comprises, in a 5′ to 3′ direction:
(a) a PCR handle sequence;
(b) a barcode sequence, wherein each barcode sequence of the polynucleotide array of each micro-particle is different to each barcode sequence of the polynucleotide array of each other micro-particle; and
(c) a second ligation linker for ligation to the first ligation linker.

11. A method for synthesising a micro-particle, the method comprising:
providing (I)
(i) a plurality of polynucleotides each comprising a first ligation linker and an analyte capture region; and
(ii) a plurality of polynucleotides each comprising the first ligation linker and a hairpin sequence;
(II) providing a micro-particle, wherein the micro-particle comprises a micro-bead and an array of polynucleotides, wherein each polynucleotide of the array comprises, in a 5′ to 3′ direction:
(a) a PCR handle sequence;
(b) a barcode sequence, wherein each barcode sequence of the polynucleotide array of each micro-particle is different to each barcode sequence of the polynucleotide array of each other micro-particle; and
(c) a second ligation linker for ligation to the first ligation linker; and
(III) contacting the micro-particle with a mixture of the plurality of polynucleotides (i) and the plurality of polynucleotides (ii) under conditions suitable for ligating the first ligation linker to the second ligation linker.

12. A method for synthesising a plurality of micro-particles according to claim 11, the method comprising:
(I) providing
(i) a plurality of polynucleotides each comprising a first ligation linker and an analyte capture region; and
(ii) a plurality of polynucleotides each comprising the first ligation linker and a hairpin sequence;
(II) providing a plurality of micro-particles, wherein each micro-particle comprises a micro-bead and an array of polynucleotides, wherein each polynucleotide of an array comprises, in a 5′ to 3′ direction:
(a) a PCR handle sequence;
(b) a barcode sequence, wherein the barcode sequence of each polynucleotide in an array is the same as the barcode sequence of essentially each other polynucleotide in the array of a micro-particle; and wherein the array of polynucleotides of each micro-particle has a different barcode sequence from the array of polynucleotides of essentially each other micro-particle; and
(c) a second ligation linker for ligation to the first ligation linker; and
(III) contacting the micro-particles with a mixture of the plurality of polynucleotides (i) and the plurality of polynucleotides (ii) under conditions suitable for ligating the polynucleotides comprising the first ligation linker to polynucleotides comprising the second ligation linker.

13. The kit of claim 9 or claim 10, or the method of claim 11 or claim 12, wherein the ligation linkers are complementary click-reactive groups.

14. A micro-particle produced by the method of claim 11 or claim 13, or a plurality of micro-particles produced by the method of any one of claim 12 or claim 13.

15. A method of generating a library of analytes from a plurality of samples, the method comprising:
(I) providing a plurality of micro-particles according to claim 8, wherein the micro-particles are divided between a plurality of compartments; and wherein the samples are divided between the same compartments, such that contact is made between co-compartmentalized micro-particles, or the arrays of polynucleotides thereof, and sample;
(II) incubating the micro-particles, or the arrays of polynucleotides thereof, with the analytes of co-compartmentalized samples under conditions suitable to allow binding between the sample analytes and the analyte capture regions of the arrays of polynucleotides, such that sample analytes are captured for the library; and
(III) incubating the micro-particles, or the arrays of polynucleotides thereof, under conditions suitable to allow different polynucleotides of the arrays comprising hairpin sequences to dimerise by annealing their complementary hairpin sequences;
wherein (II) and (III) may be conducted in either order, or combined together.

16. The method of claim 15, further comprising releasing the arrays of polynucleotides from the micro-beads into the compartments, wherein the polynucleotides may be released from the micro-beads at, or in between, any of steps (I) to (III).

17. The method of claim 16, wherein step (I) comprises providing a plurality of micro-particles according to claim 8, and wherein the arrays of polynucleotides are released from the micro-beads by cleaving the linker, optionally by exposure to UV light.

18. The method of any one of claims 15 to 17, wherein step (III) comprises heating the compartments to a temperature suitable for denaturing the hairpin structures; and cooling the compartments to a temperature suitable for different polynucleotides comprising the hairpin sequence to dimerise.

19. The method of any one of claims 15 to 18, further comprising filling in the single stranded overhangs of the dimerised polynucleotides to produce a set of double stranded polynucleotides comprising a hairpin sequence flanked by two barcode sequences.

20. The method of claim 19, wherein the method comprises reverse transcription using the free 5′ ends of the dimers as template to produce the double stranded polynucleotides comprising a hairpin sequence and two barcode sequences.

21. The method of any one of claims 15 to 20, further comprising combining the plurality of compartments.

22. The method of any one of claims 15 to 21, further comprising removing polynucleotides comprising the hairpin sequence; optionally wherein the non-dimerised polynucleotides are removed using a single-strand specific endo- or exonuclease.

23. The method of any one of claims 15 to 22, further comprising amplifying the polynucleotides comprising a hairpin sequence and two barcode sequences.

24. The method of any one of claims 15 to 23, further comprising separating the analyte capture polynucleotides, the analyte-bound polynucleotides, and/or the amplified products thereof, from the polynucleotides comprising a hairpin sequence and two barcode sequences.

25. The method of any one of claims 15 to 24, further comprising sequencing the polynucleotides comprising a hairpin sequence and two barcode sequences.

26. The method of any one of claims 15 to 25, further comprising identifying a captured analyte from a sample that was co-compartmentalized during library generation with multiple micro-particles, or the arrays of polynucleotides thereof, the method comprising:
(I) identifying a polynucleotide that was produced by the method of any one of claims 15 to 25, and that comprises the hairpin sequence and two different barcode sequences;
(II) identifying an analyte that was captured by a polynucleotide that shares either of the two barcode sequences with the polynucleotide identified in step (I); and
(III) identifying the captured analyte identified in step (II) as being from a sample that was co-compartmentalized during library generation with multiple micro-particles, or the arrays of polynucleotides thereof.

27. The method of any one of claims 15 to 25, further comprising identifying a captured analyte from a sample that was co-compartmentalized during library generation with two different micro-particles only, or the arrays of polynucleotides thereof, the method comprising:
(I) identifying a polynucleotide that was produced by the method of any one of claims 15 to 25, and that comprises the hairpin sequence and two different barcode sequences;
(II) identifying all, or substantially or essentially all of the polynucleotide that comprise the hairpin sequence and either of the two barcode sequences of the polynucleotide identified in step (I);
(III) determining that all of the polynucleotides identified in step (II) do not comprise any barcode sequence other than the two barcode sequences of the polynucleotide identified in step (I);
(IV) identifying an analyte captured by a polypeptide that shares the either one of the two barcode sequences as the polynucleotides identified in steps (I) and (II); and
(V) identifying the analyte identified in step (IV) as being from a sample that was co-compartmentalized during library generation with two micro-particle only, or the arrays of polynucleotides thereof.

28. The method of any one of claims 15 to 26, further comprising identifying a set of captured analytes from sample that was co-compartmentalized during library generation with multiple micro-particles, or the arrays of polynucleotides thereof, the method comprising:
(I) identifying a set of polynucleotides that were produced by the method of any one of claims 15 to 25 and that each comprise the hairpin sequence and two different barcode sequences, wherein all of said barcode sequences in the set of polynucleotides share a string of different combinations of the barcode sequences between the polynucleotides of the set;
(II) identifying a set of analytes captured by polynucleotides that share any of the barcode sequences with the set of polynucleotides identified in step (I); and
(III) identifying the set of analytes identified in step (II) as being from sample that was co-compartmentalized during library generation with multiple micro-particles, or the arrays of polynucleotides thereof.

29. The method of any one of claims 15 to 25, further comprising identifying a captured analyte from sample that was co-compartmentalized during library generation with one micro-particle only, or the arrays of polynucleotides thereof, the method comprising:
(I) identifying a polynucleotide that was produced by the method of any one of claims 15 to 25, and that comprises the hairpin sequence and two identical barcode sequences;
(II) identifying all of the polynucleotide that were produced by the method of any one of claims 15 to 25, and that comprise the hairpin sequence and barcode sequence of the polynucleotide identified in step (I);
(III) determining that all of the polynucleotides identified in step (II) comprise two copies of the same barcode sequence;
(IV) identifying an analyte captured by a polynucleotide that shares the same barcode sequence as the polynucleotides identified in steps (I) and (II); and
(V) identifying the analyte identified in step (IV) as being from a sample that was co-compartmentalized during library generation with one micro-particle only, or the arrays of polynucleotides thereof.


SEQUENCE LISTING (20 sequences)

SEQ ID NO: 1 - DNA, 25 bp, synthetic construct
  source 1..25: mol_type = other DNA
aagcagtggtatcaacgcagagtac

SEQ ID NO: 2 - DNA, 21 bp, synthetic construct
  source 1..21: mol_type = other DNA
tacacgacgctcttccgatct

SEQ ID NO: 3 - DNA, 12 bp, synthetic construct
  source 1..12: mol_type = other DNA
aaggccttaagg

SEQ ID NO: 4 - DNA, 20 bp, synthetic construct
  source 1..20: mol_type = other DNA
gtggtatcaacgcagagtac

SEQ ID NO: 5 - DNA, 20 bp, synthetic construct
  source 1..20: mol_type = other DNA
gtccgagcgtaggttatccg

SEQ ID NO: 6 - DNA, 65 bp, synthetic construct
  source 1..65: mol_type = other DNA
aagcagtggtatcaacgcagagtacgcccgggccagannnnnnnngacttaagaattcttaagtc

SEQ ID NO: 7 - DNA, 57 bp, synthetic construct
  source 1..57: mol_type = other DNA
aagcagtggtatcaacgcagagtacagattgggacgcnnnnnnnngacttataagtc

SEQ ID NO: 8 - DNA, 77 bp, synthetic construct
  source 1..77: mol_type = other DNA
aagcagtggtatcaacgcagagtacgcaagaaaaaatcacgacttgtaagtggttacnnnnnnnngacttataagtc

SEQ ID NO: 9 - DNA, 65 bp, synthetic construct
  source 1..65: mol_type = other DNA
aagcagtggtatcaacgcagagtacagattgggacgcnnnnnnnngacttaagaattcttaagtc

SEQ ID NO: 10 - DNA, 85 bp, synthetic construct
  source 1..85: mol_type = other DNA
aagcagtggtatcaacgcagagtacgcaagaaaaaatcacgacttgtaagtggttacnnnnnnnngacttaagaattcttaagtc

SEQ ID NO: 11 - DNA, 23 bp, synthetic construct
  source 1..23: mol_type = other DNA
aagcagtggtatcaacgcagagt

SEQ ID NO: 12 - DNA, 54 bp, synthetic construct
  source 1..54: mol_type = other DNA
tctctctaagcagtggtatcaacgcagagtacnnnnnnnnnnnnggtgtaatga

SEQ ID NO: 13 - DNA, 65 bp, synthetic construct
  source 1..65: mol_type = other DNA
ggtcgtagtgnnnnnnnnnnnnnnnnnnnnnnnnvtttttttttttttttttttttttttttttt

SEQ ID NO: 14 - DNA, 20 bp, synthetic construct
  source 1..20: mol_type = other DNA
cactacgacctcattacacc

SEQ ID NO: 15 - DNA, 47 bp, synthetic construct
  source 1..47: mol_type = other DNA
ggtcgnnnnnnnnacgatttttttttttttttttttttttttttttt

SEQ ID NO: 16 - DNA, 25 bp, synthetic construct
  source 1..25: mol_type = other DNA
ggtcggacttaagaattcttaagtc

SEQ ID NO: 17 - DNA, 46 bp, synthetic construct
  source 1..46: mol_type = other DNA
tctctctctacacgacgctcttccgatctnnnnnnnnnnnnaatga

SEQ ID NO: 18 - DNA, 10 bp, synthetic construct
  source 1..10: mol_type = other DNA
ttactccagc

SEQ ID NO: 19 - DNA, 93 bp, synthetic construct
  source 1..93: mol_type = other DNA
tctctctctacacgacgctcttccgatctnnnnnnnnnnnnaatgaggtcgnnnnnnnnacgatttttttttttttttttttttttttttttt

SEQ ID NO: 20 - DNA, 71 bp, synthetic construct
  source 1..71: mol_type = other DNA
tctctctctacacgacgctcttccgatctnnnnnnnnnnnnaatgaggtcggacttaagaattcttaagtc